(19) United States
(12) Patent Application Publication
(10) Pub. No.: US 2026/0266840 A1
(43) Pub. Date: Sep. 10, 2026

(54) METHODS AND COMPOSITIONS FOR DIANGOSIS OF ECTOPIC PREGNANCY

(71) Applicants: The Wistar Institute of Anatomy and Biology, Philadelphia, PA (US); The Trustees of the University of Pennsylvania, Philadelphia, PA (US)

(72) Inventors: David W. Speicher, Berwyn, PA (US); Kurt T. Barnhart, Bryn Mawr, PA (US); Aaron R. Goldman, Philadelphia, PA (US)

(21) Appl. No.: 19/166,380

(22) PCT Filed: Mar. 18, 2024
(86) PCT No.: PCT/US2024/020414

§ 371 Date: Sep. 17, 2025

Related U.S. Application Data
(60) Provisional application No. 63/491,009, filed on Mar. 17, 2023.

(51) Int. Cl. G01N 33/68; G01N 33/543

(52) U.S. Cl. CPC G01N 33/689; G01N 33/543; G01N 2333/471; G01N 2333/918; G01N 2333/96419; G01N 2800/368; G01N 2800/60

(57) ABSTRACT

Methods and compositions are provided for diagnosing ectopic pregnancy or nonviable pregnancy in a mammalian subject by detecting changes in expression of the expression levels of one or more of proteins or peptide fragments.

### CROSS-REFERENCE TO RELATED APPLICATIONS

This application claims priority of U.S. Provisional application No. 63/491,009 filed Mar. 17, 2023, the entire contents being incorporated herein by reference as though set forth in full.

### STATEMENT REGARDING FEDERALLY SPONSORED RESEARCH OR DEVELOPMENT

This invention was made with government support under grant numbers HD076279 and CA010815 awarded by the National Institutes of Health. The government has certain rights in the invention.

### INCORPORATION-BY-REFERENCE OF MATERIAL SUBMITTED IN ELECTRONIC FORM

The Contents of the electronic sequence listing (WST-196PCT_SEQLIST.xml; Size: 32,013 bytes; and Date of Creation: Mar. 18, 2024) is herein incorporated by reference in its entirety.

### BACKGROUND

In a field of consistently improving techniques and technologies, the distinction between a normal versus abnormal early pregnancy remains a vexing clinical challenge for health care providers and their female patients. The importance of being able to accurately make the diagnosis of either a viable intrauterine pregnancy (IUP), pregnancy loss (EPL), or ectopic pregnancy (EP) cannot be overstated, with each of these outcomes necessitating unique treatment pathways as well as contributing strikingly different rates of maternal morbidity and mortality.

Distinguishing a normal intrauterine pregnancy (IUP) from abnormal gestations is a clinical challenge because there is no definitive noninvasive diagnostic test when ultrasound is non-diagnostic. Pregnant women presenting with lower abdominal pain and/or vaginal bleeding will ultimately be diagnosed with: a viable IUP, a miscarriage or early pregnancy loss (EPL), or an ectopic pregnancy (EP). EP occurs in 1-2% of pregnant women and is a leading cause of maternal mortality and morbidity, accounting for 6% of pregnancy-related deaths [1,2], whereas EPL affects 10%-20% of pregnancies [3]. Clinical management of these three outcomes is drastically different, and a timely and accurate diagnosis is imperative because undiagnosed EP can be fatal. Currently, serial β-human chorionic gonadotropin (β-hCG, gene name: CGB) and progesterone are the most widely used serum biomarkers for evaluating pregnancy outcome when ultrasound is inconclusive [4,5]. Likewise, serial β-hCG levels and pelvic ultrasound are the standard methods of diagnosing an EP. However, neither method can definitively diagnose an EP with sufficient and reproducible accuracy at very early stages [2, 6].

In reproductive medicine, biomarker development has become a rapidly evolving field channeled towards conditions that affect both acute and long-term maternal health. In conditions with the potential for the highest levels of morbidity and mortality such as ectopic pregnancy (EP), biomarkers can change the way we diagnosis and manage women with a possible abnormal early pregnancy. The use of new markers can further improve accuracy and discrimination ultimately leading to widespread clinical use.

What is needed is improved compositions and methods for diagnosing ectopic pregnancies.

### SUMMARY OF THE INVENTION

In one aspect, the invention includes diagnostic reagents or kits for use in diagnosing an ectopic pregnancy in a mammalian subject comprising: (a) a ligand that that binds to a peptide or protein selected from the group consisting of: i. disintegrin and metalloproteinase domain-containing protein 12 (ADAM12), ii. Glycoprotein hormones alpha chain (CGA), iii. Choriogonadotropin subunit beta (CGB), iv. Isthmin-2 (ISM2), v. protein notum homolog (NOTUM), vi. Glycodelin (PAEP), vii. Pappalysin-1 (PAPPA), viii. pregnancy specific beta-1 glycoprotein, isoform 1 (PSG1), ix. pregnancy specific beta-1 glycoprotein, isoform 2 (PSG2), x. pregnancy specific beta-1 glycoprotein, isoform 3 (PSG3), xi. pregnancy specific beta-1 glycoprotein, isoform 9 (PSG9), xii. pregnancy specific beta-1 glycoprotein, isoform 11 (PSG11), xiii. pregnancy specific beta-1 glycoprotein, isoforms 6 and 9 (PSG6/9), and xiv. pregnancy specific beta-1 glycoprotein, isoforms 8 and 1 (PSG8/1), or (b) a combination of ligand (a), wherein each ligand binds to a different peptide or protein (i) through (xiv); wherein at least one of the ligands is associated with a detectable label or with a substrate.

In certain embodiments, the above reagent or kit consist of (v), (vi), (vii) and (ix). In another embodiment the above reagent or kit consists of (iv), (vi), (vii) and (ix). In another embodiment the above reagent or kit consists of (iv), (vi), (vii) and (i). In certain embodiment the above reagent or kit consists of (v), (vi), (vii) and (i).

In certain embodiments, the kit or reagent comprises a substrate upon which said ligand is immobilized. In another aspect, ligand is associated with a detectable label. In other embodiments, said ligands or said expression products are proteins or peptides. In certain embodiments, said ligand is an antibody or fragment thereof. In certain embodiments, the kit or reagent further comprises a microarray, a microfluidics card, a chip or a chamber.

In another aspect, the invention includes methods for diagnosing an ectopic pregnancy in a female mammalian subject comprising (a) measuring in a biological fluid sample of the subject the expression level of a protein or peptide fragment thereof selected from the group consisting of: i. disintegrin and metalloproteinase domain-containing protein 12 (ADAM12), ii. Glycoprotein hormones alpha chain (CGA), iii. Choriogonadotropin subunit beta (CGB), iv. Isthmin-2 (ISM2), v. protein notum homolog (NOTUM), vi. Glycodelin (PAEP), vii. Pappalysin-1 (PAPPA), viii. pregnancy specific beta-1 glycoprotein, isoform 1 (PSG1), ix. pregnancy specific beta-1 glycoprotein, isoform 2 (PSG2), x. pregnancy specific beta-1 glycoprotein, isoform 3 (PSG3), xi. pregnancy specific beta-1 glycoprotein, isoform 9 (PSG9), xii. pregnancy specific beta-1 glycoprotein, isoform 11 (PSG11), xiii. pregnancy specific beta-1 glycoprotein, isoforms 6 and 9 (PSG6/9), and xiv. pregnancy specific beta-1 glycoprotein, isoforms 8 and 1 (PSG8/1), and (b) comparing said subject's expression level of the selected protein or peptide fragment with the level of the same protein or peptide in the biological fluid of a reference or control female mammalian subject having a normal intrauterine pregnancy (IUP), wherein changes in expression of the subject's selected protein or peptide fragment from those of the reference or control correlates with a diagnosis of ectopic pregnancy.

In certain embodiments, said change in expression level of each said selected protein or peptide fragment comprises an upregulation in comparison to said reference or control or a downregulation in comparison to said reference or control.

In certain embodiments, the method comprises measuring biomarkers (v), (vi), (vii) and (ix). In another embodiment, biomarkers (iv), (vi), (vii) and (ix) are measured. In another embodiment biomarkers (iv), (vi), (vii) and (i) are measured. In certain embodiment biomarkers (v), (vi), (vii) and (i) are measured.

Other aspects and advantages of the invention will be readily apparent from the following detailed description of the invention.

### DESCRIPTION OF THE FIGURES

FIG. 1: Scheme for discovery and validation of candidate biomarkers. Candidate biomarkers were identified by LC-MS/MS using label-free quantitation in a discovery cohort of 48 pregnant women. Biomarkers were then validated with targeted PRM-MS in an independent cohort of 74 women.

FIG. 2A-2B: Identification of candidate biomarkers in the discovery cohort. FIG. 2A. Volcano plot for EP vs non-EP (IUP+EPL) FIG. 2B. Volcano plot comparing EPL vs IUP. High priority biomarkers outside the curves (FDR≤0.05) are highlighted in red. Additional proteins having p≤0.05 and fold change ≥3 are represented by black circles. Labeled proteins were further investigated in the validation cohort.

FIG. 3A-3B: Scatterplots of candidate EP vs non-EP biomarkers from the discovery cohort. Biomarkers were selected from volcano plots in FIG. 2A for further validation. FIG. 3A. High-priority (FDR≤0.05) FIG. 3B. Additional candidate markers (p≤0.05 and fold change ≥3). Wilcoxon rank sum test was used to compare EP vs non-EP (IUP+EPL) and statistical significance is shown above brackets (*<0.05, **<0.01, ***<0.001, n.s.=not significant). For visualization, zero values are plotted on the x-axis.

FIG. 4A-4C: Scatterplots of candidate EP vs non-EP biomarkers from the validation cohort. Wilcoxon rank sum test was used to compare EP vs non-EP (IUP+EPL) and statistical significance is shown above brackets (*<0.05, **<0.01, ***<0.001). For visualization, zero values are plotted on the x-axis.

FIG. 5: Correlation of predictors of EP vs non-EP. Cluster dendrogram based on Spearman correlation. Lasso-selected protein markers (NOTUM, PAEP, PAPPA, and PSG2) are noted with red asterisks.

FIG. 6A-6D: Prediction ability of multivariable logistics models for EP vs non-EP. FIG. 6A. Scatterplot showing the risk score of having EP by each group for the multivariable logistics model with predictors selected from Lasso (Model 1). FIG. 6B. prediction ability of Model 2 (using ISM2 instead of NOTUM). FIG. 6C. Prediction ability of Model 3 (using ISM2 instead of NOTUM, and using ADAM12 instead of PSG2). FIG. 6D. Prediction ability of Model 4 (using ADAM12 instead of PSG2).

FIG. 7A-7E: is a table showing LFQ for selected EP vs non-EP candidate biomarkers.

### DETAILED DESCRIPTION OF THE INVENTION

Proteomics-based discovery of serum/plasma biomarkers of early pregnancy complications as well as other clinical disorders is feasible but challenging because: 1) plasma and serum proteomes are very complex; 2) abundant plasma proteins that are present in the mg/ml range severely limit detection of lower abundance proteins; and 3) most clinical biomarkers are present in the ng/ml to pg/ml range or even lower [7-9]. These challenges have been partially addressed by major advances in liquid chromatography-tandem mass spectrometry (LC-MS/MS) instrumentation and methodologies.

Proteomics discoveries of serum/plasma typically identify moderate numbers (>10) of candidate biomarkers that need to be validated in independent clinical patient cohorts to determine which biomarkers are likely to have sufficient diagnostic accuracy. A major gap exists between candidate biomarker discovery and development of a commercial biomarker assay because, typically, robust, high sensitivity quantitative assays for newly discovered biomarkers are not available. Further, antibody-based assays are difficult to multiplex for high-throughput analysis of candidate biomarker panels. To bridge this gap, we developed a parallel reaction monitoring mass spectrometry (PRM-MS)-based targeted assay that distinguishes between candidate early pregnancy biomarkers. Using this assay, we determined the minimum number of protein biomarkers needed for accurate EP diagnosis and demonstrated that this panel of biomarkers can accurately distinguish an EP from other pregnancy outcomes.

It is to be noted that the term “a” or “an” as used herein refers to one or more. As such, the terms “a” (or “an”), “one or more,” and “at least one” are used interchangeably herein.

As used herein, the term “about” means a variability of 10% from the reference given, unless otherwise specified.

“Patient” or “subject” as used herein means a female mammalian animal, including a human, a veterinary or farm animal, a domestic animal or pet, and animals normally used for clinical research. In one embodiment, the subject of these methods and compositions is a human.

“Control” or “Control subject” as used herein refers to both an individual female with IUP or the pooled biological fluids (e.g., sera) from multiple females with IUP or numerical or graphical averages of the expression levels of the selected biomarkers obtained from large groups of females with IUP. Such controls are the types that are commonly used in similar diagnostic assays for other biomarkers. Selection of the particular class of controls depends upon the use to which the diagnostic methods and compositions are to be put by the physician. As used herein, the term “predetermined control” refers to a numerical level, average, mean or average range of the expression of a biomarker in a defined population. The predetermined control level is preferably provided by using the same assay technique as is used for measurement of the subject's biomarker levels, to avoid any error in standardization. For example, the control may comprise a single healthy pregnant mammalian subject at the same time of pregnancy as the subject. In another embodiment, the control comprises a population of multiple healthy pregnant mammalian subjects at the same time of pregnancy as the subject or multiple healthy IUP mammalian subjects. In another embodiment, the control comprises the same subject at an earlier time in the pregnancy. In addition, a predetermined control may also be a negative predetermined control. In one embodiment, a negative predetermined control comprises one or multiple subjects who have EP. The control can refer to a numerical average, mean or average range of the expression of one or more biomarkers, in a defined population, rather than a single subject.

The phrase “intrauterine pregnancy” or “IUP” or “viable pregnancy” refers to a pregnancy that results in a live birth or confirmed second trimester viability.

The phrase “ectopic pregnancy” or “EP” refers to a pregnancy in which the fetus develops outside of the uterus. Typically, implantation occurs in the fallopian tube. Ectopic pregnancy generally requires surgical removal of the fetus within 48 hours of diagnosis.

The phrase “non-viable pregnancy”, “pregnancy loss”, “early pregnancy loss”, “EPL”, “spontaneous abortion”, and “SAB” refers to an intrauterine pregnancy that cannot possibly result in a liveborn baby. Non-viable pregnancy includes fetal demise, where the is no fetal cardiac activity within the first 7 weeks of pregnancy, and anembryonic gestation, where no fetus is present in the gestational sac.

“Sample” as used herein means any biological fluid or tissue that contains the biomarkers. The most suitable samples for use in the methods and with the compositions are blood samples, including serum, plasma, whole blood, and peripheral blood. It is also anticipated that other biological fluids, such as saliva or urine, vaginal or cervical secretions, amniotic fluid, and placental fluid may be used similarly. Such samples may further be diluted with saline, buffer or a physiologically acceptable diluent. Alternatively, such samples are concentrated by conventional means.

By “change in expression” is meant an increased expression level of a selected biomarker, or upregulation of the genes or transcript encoding it in comparison to the reference or control; a decreased expression level of a selected biomarker or a downregulation of the genes or transcript encoding it in comparison to the reference or control; or a combination of certain increased/upregulated and decreased/down regulated biomarkers. The degree of change in target expression can vary with each individual and is subject to variation with each population and days or weeks of the pregnancy. For example, in one embodiment, a large change, e.g., 2-3 fold increase or decrease in a small number of biomarkers, e.g., from 1 to 9 characteristic biomarkers, is statistically significant. In another embodiment, a smaller relative change in about 3, 4, 5, 6, 7, 8, 9, 10, 11, 12, 13, 14, or more biomarkers is statistically significant.

By “target biomarker” or “target biomarker signature” as used herein is meant those proteins/peptides or the genes/transcripts encoding same, the expression of which changes (either in an up-regulated or down-regulated manner) characteristically in the presence of an ectopic pregnancy from that in an IUP or EPL. In one embodiment, at least one target biomarker forms a suitable biomarker signature for use in the methods and compositions. In one embodiment, at least two target biomarkers form a suitable biomarker signature for use in the methods and compositions. Specific biomarker signatures can include any combination of EP biomarkers employing at least one biomarker from (i) to (xiv) identified herein and including all biomarker combinations in Table 1, as well as other combinations with the biomarkers. One skilled in the art may readily reproduce the compositions and methods described herein by use of the sequences of the biomarkers, all of which are publicly available from conventional sources, such as GenBank. As used herein, “target biomarkers” refers to the full length, or a portion of the identified protein or coding sequence.

The term “microarray” refers to an ordered arrangement of hybridizable array elements, e.g., primers, probes, ligands, on a substrate.

The term “ligand” refers to a molecule that binds to a protein or peptide, and includes antibodies and fragments thereof.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Useful Target Biomarkers for Determining Pregnancy Location and Viability

The “targets” of the compositions and methods of these inventions include, in one aspect, the genes, gene fragments, transcripts and the expression products, including the groups of proteins and peptide fragments thereof, listed in Table 1. As described in the Examples below, the inventors identified 14 proteins that differed in expression between the conditions of EP and EPL and IUP. Further analysis resulted in the identification of highly significant protein biomarkers that can reliably distinguish between the conditions of EP and EPL and IUP. In certain embodiments, the compositions and methods for distinguishing ectopic pregnancy from normal intrauterine pregnancy utilize at least one of the novel biomarkers, or one of the specifically identified isoforms or fragments of known markers. In other embodiments, the compositions and methods for distinguishing ectopic pregnancy from normal intrauterine pregnancy utilize at least two or more of the specific target biomarker protein forms identified herein. In still other embodiments, the compositions and methods for distinguishing ectopic pregnancy from normal intrauterine pregnancy will utilize at least three or more of the specific target biomarker protein forms identified herein. In still other embodiments, the compositions and methods for distinguishing ectopic pregnancy from normal intrauterine pregnancy will utilize at least five or more of the specific target biomarker protein forms identified herein. In still other embodiments, at least 14 or more biomarkers will be employed in the methods and compositions described herein for diagnosis of EP.

TABLE 1
Biomarkers useful in the compositions and methods

| | | | |
|---|---|---|---|
| i. | ADAM12 | ii. | CGA |
| iii. | CGB | iv. | ISM2 |
| v. | NOTUM | vi. | PAEP |
| vii. | PAPPA | viii. | PSG1 |
| ix. | PSG2 | x. | PSG3 |
| xi. | PSG9 | xii. | PSG11 |
| xiii. | PSG6 | xiv. | PSG8 |

In one embodiment a target of the methods and compositions described herein is ADAM12, known as disintegrin and metalloproteinase domain-containing protein 12. The amino acid sequence and nucleic acid sequence for ADAM12 are publicly available, see, e.g., GENBANK Accession No. NP_001275902.1 and NM_001288973.2.

In another embodiment a target of the methods and compositions described herein is CGA, known as Glycoprotein hormones alpha chain. The amino acid sequence and nucleic acid sequence for CGA are publicly available, see, e.g., GENBANK Accession No. NP_001239312.1 and NM_001252383.2.

In another embodiment a target of the methods and compositions described herein is CGB, known as Choriogonadotropin subunit beta. The amino acid sequence and nucleic acid sequence for CGB are publicly available, see, e.g., GENBANK Accession No. NP_203695.2 and NM_033377.2.

In another embodiment a target of the methods and compositions described herein is ISM2, known as Isthmin-2. The amino acid sequence and nucleic acid sequence for ISM2 are publicly available, see, e.g., GENBANK Accession No. NP_954993.1 and NM_199296.3.

In another embodiment a target of the methods and compositions described herein is NOTUM, known as notum, palmitoleoyl-protein carboxylesterase. The amino acid sequence and nucleic acid sequence for NOTUM are publicly available, see, e.g., GENBANK Accession No. NP_848588.3 and NM_178493.6.

In another embodiment a target of the methods and compositions described herein is PAEP, known as Glycodelin. The amino acid sequence and nucleic acid sequence for PAEP are publicly available, see, e.g., GENBANK Accession No. NP_002562.2 and NM_001018049.3.

In another embodiment a target of the methods and compositions described herein is PAPPA, known as Pappalysin-1. The amino acid sequence and nucleic acid sequence for PAPPA are publicly available, see, e.g., GENBANK Accession No. NP_002572.2 and NM_002581.5.

In another embodiment a target of the methods and compositions described herein are specific isoforms of a family of related proteins produced by the placenta, called pregnancy specific beta-1 glycoprotein (PSG; also called serum specific protein-1 (SP1)). The inventors determined that certain isoforms not previously associated with EP can be used as biomarkers/targets in the methods and compositions described herein. Thus, in one embodiment a target for use herein is PSG, isoform 1 (PSG1). The amino acid sequence and nucleic acid sequence for PSG1 are publicly available, see, e.g., GENBANK Accession No. NM_006905.3 and XP_005259122.1.

In another embodiment, a target for use herein is PSG, isoform 2 (PSG2). The amino acid sequence and nucleotide sequence for PSG2 are publicly available, see, e.g., GENBANK Accession No. NP_112536.2 and NM_031246.4. In another embodiment, a target for use herein is PSG, isoform 3 (PSG3). The amino acid sequence and nucleotide sequence for PSG3 are publicly available, see, e.g., GENBANK Accession No. NM_021016.4 and NP_066296.2.

In another embodiment a target for use herein is PSG, isoform 9 (PSG9). The amino acid sequence and nucleotide sequence for PSG9 are publicly available, see, e.g., GENBANK Accession No. NP_002775.3 and NM_002784.5. In another embodiment a target for use herein is PSG, isoform 11 (PSG11). The amino acid sequence and nucleotide sequence for PSG11 are publicly available, see, e.g., GENBANK Accession No. NP_002776.3 and NM_002785.3.

In another embodiment a target for use herein is PSG, isoform 6 (PSG6). The amino acid sequence and nucleotide sequence for PSG6 are publicly available, see, e.g., GENBANK Accession No. NP_002773.1 and NM_002782.5. In certain embodiments, the peptide marker for PSG6 is shared with PSG9. Said marker is identified as PSG6/9.

In another embodiment a target for use herein is PSG, isoform 8 (PSG8). The amino acid sequence and nucleotide sequence for PSG8 are publicly available, see, e.g., GENBANK Accession No. NP_874366.1 and NM_182707.3. In certain embodiments, the peptide marker for PSG8 is shared with PSG1. Said marker is identified as PSG8/1.

In still other embodiments, the target for use in the methods and compositions described herein can include various combinations of these target biomarkers and/or fragments thereof.

Among desirable biomarker signatures for the discrimination of pregnancy location are signatures including at least one biomarker, at least two biomarkers, at least three biomarkers, at least five biomarkers, at least 6, at least 7, at least 8, at least 9, at least 10, at least 11, at least 12, at least 13, or all 14 biomarkers selected from ADAM12, CGA, CGB, ISM2, NOTUM, PAEP, PAPPA, PSG1, PSG2, PSG3, PSG9, PSG11, PSG6/9, and PSG8/1. Other suitable biomarker signatures include combinations of at least one of the above noted 14 biomarkers with at least one additional biomarker. In another embodiment, the biomarkers comprise CGB, glycoprotein hormones alpha chain (CGA), isthmin-2 (ISM2), glycodelin (PAEP), pregnancy-specific beta-1-glycoprotein 1 (PSG1), pregnancy-specific beta-1-glycoprotein 2 (PSG2), pregnancy-specific beta-1-glycoprotein 3 (PSG3), and pregnancy-specific beta-1-glycoprotein 9 (PSG9). In another embodiment, the biomarkers comprise PAEP, PSG9, PSG3, CGB, ISM2, PSG2, CGA, PSG1, NOTUM, PAPPA, PSG11, and ADAM12. In another embodiment, the biomarkers comprise NOTUM, PAEP, PAPPA, and PSG2. In another embodiment, the biomarkers comprise ISM2, PAEP, PAPPA, and PSG2. In another embodiment, the biomarkers comprise NOTUM, PAEP, PAPPA, and ADAM12. In another embodiment, the biomarkers comprise ISM2, PAEP, PAPPA, and ADAM12. Still another embodiment of a biomarker signature contains all of the biomarkers of Table 1.

### Diagnostic Reagents and Kits

### Labeled or Immobilized Biomarkers or Peptides

In one embodiment, diagnostic reagents for use in the methods of determining pregnancy location includes one target biomarker identified herein, associated with a detectable label or portion of a detectable label system. In another embodiment, a diagnostic reagent includes one target biomarker herein, immobilized on a substrate. In still another embodiment, combinations of such labeled or immobilized biomarkers are suitable reagents and components of a diagnostic kit. Among such immobilized or labeled biomarkers are those selected from the biomarkers of Table 1.

In another aspect, suitable embodiments of such labeled or immobilized reagents include at least one, 2, 3, 4, 5, 6, 7, 8, 9, 10, 11, 12, 13, or all 14 of biomarkers (i) to (xiv) of Table 1, or their unique peptide fragments therein.

### LC-MS/MS

LC-MS/MS combines the physical separation of liquid chromatography (including high performance liquid chromatography) with the mass analysis of mass spectrometry (MS). LC-MS/MS has high sensitivity and selectivity and is useful for separation, detection and identification of proteins.

Protein quantification by LC-MS/MS usually includes proteolytic digestion of the analyte with an enzyme such as trypsin, to cleave it into a set of smaller peptides, one of which, the signature peptide, is subsequently used for quantification as a surrogate for the protein. In certain embodiments, diagnostic reagents are the surrogate peptides used for an LC-MS/MS assay. LC-MS/MS combines the separating power of liquid chromatography with the highly sensitive and selective mass analysis of mass spectrometry. In certain embodiments, a hybrid quadrupole-Orbitrap mass spectrometer is used for PRM analysis. The procedures for performing LC-MS/MS are well known to the skilled artisan (see e.g., US2019/0369116A1). In certain embodiments, the surrogate peptide is one identified in Table 5.

The LC-MS/MS quantifies the biomarkers with a high degree of sensitivity and selectivity based on the unique mass/charge (m/z) transitions of each compound. This allows for a quantitative measurement of each biomarker in the solution.

The reagents and kits discussed herein may also be useful in “parallel reaction monitoring” or “PRM”. As discussed herein, “parallel reaction monitoring” or “PRM” refers to a targeted proteomics technology used to quantify a plurality of proteins/peptides in the same experiment. In PRM, all fragment ions instead of only selected ones are measured after fragmentation of a selected precursor. PRM is typically performed on Orbitrap or Time of flight (ToF) analyzer. PRM can reduce assay development time because no target transitions (product ions) need to be preselected. PRM eliminates most interferences, providing improved accuracy and attomole-level limits of detection and quantification.

Any combination of labeled or immobilized biomarkers can be assembled in a diagnostic kit for the purposes of diagnosing EP. For example, one embodiment of a diagnostic kit includes labeled or immobilized reagents described in Table 1. Still other components of the biomarker signatures, associated with detectable labels or immobilized on substrates provide additional diagnostic kits. Still other components of the biomarker signatures are labeled or immobilized biomarkers or fragments thereof as listed in Table 1.

In certain embodiments, a combination of biomarkers includes NOTUM, PAEP, PAPPA, and PSG2. In other embodiments, a combination of biomarkers includes ISM2, PAEP, PAPPA, and PSG2. In other embodiments, a combination of biomarkers includes ISM2, PAEP, PAPPA, and ADAM12. In certain embodiments, a combination of biomarkers includes NOTUM, PAEP, PAPPA, and ADAM12. See, Table 2.

TABLE 2
Multivariable logistic regression models for EP vs non-EP (N = 74)

| | | | | | |
|---|---|---|---|---|---|
| | | | | | 95% Confidence |
| Predictors | Coefficient | SE | z | P > \|z\| | interval |

Model 1: NOTUM + PAEP + PAPPA + PSG2

| | | | | | | |
|---|---|---|---|---|---|---|
| NOTUM | −1.62 | 0.865 | −1.87 | 0.061 | −3.316 | 0.076 |
| PAEP | −1.573 | 0.535 | −2.94 | 0.003 | −2.622 | −0.524 |
| PAPPA | −0.03 | 0.197 | −0.15 | 0.878 | −0.417 | 0.356 |
| PSG2 | 0.097 | 0.52 | 0.19 | 0.853 | −0.923 | 1.117 |
| Constant | 13.282 | 4.562 | 2.91 | 0.004 | 4.341 | 22.223 |

Model 2: ISM2 + PAEP + PAPPA + PSG2

| | | | | | | |
|---|---|---|---|---|---|---|
| ISM2 | −0.31 | 0.386 | −0.8 | 0.422 | −1.066 | 0.447 |
| PAEP | −1.349 | 0.444 | −3.04 | 0.002 | −2.22 | −0.478 |
| PAPPA | −0.109 | 0.174 | −0.63 | 0.531 | −0.45 | 0.232 |
| PSG2 | −0.338 | 0.476 | −0.71 | 0.477 | −1.27 | 0.594 |
| Constant | 8.312 | 2.881 | 2.89 | 0.004 | 2.665 | 13.958 |

Model 3: ISM2 + PAEP + PAPPA + ADAM12

| | | | | | | |
|---|---|---|---|---|---|---|
| ISM2 | −0.336 | 0.421 | −0.8 | 0.424 | −1.161 | 0.488 |
| PAEP | −1.349 | 0.443 | −3.05 | 0.002 | −2.216 | −0.482 |
| PAPPA | −0.141 | 0.163 | −0.86 | 0.387 | −0.462 | 0.179 |
| ADAM12 | −0.236 | 0.503 | −0.47 | 0.639 | −1.223 | 0.75 |
| Constant | 8.43 | 2.944 | 2.86 | 0.004 | 2.66 | 14.2 |

Model 4: NOTUM + PAEP + PAPPA + ADAM12

| | | | | | | |
|---|---|---|---|---|---|---|
| NOTUM | −2.201 | 1.159 | −1.9 | 0.058 | −4.474 | 0.071 |
| PAEP | −1.739 | 0.622 | −2.8 | 0.005 | −2.959 | −0.52 |
| PAPPA | −0.128 | 0.232 | −0.55 | 0.58 | −0.584 | 0.327 |
| ADAM12 | 0.62 | 0.772 | 0.8 | 0.422 | −0.892 | 2.133 |
| Constant | 14.289 | 5.071 | 2.82 | 0.005 | 4.351 | 24.227 |

*Coefficients and constants are used to calculate risk scores for each individual patient.

For these reagents, the labels may be selected from among many known diagnostic labels, including those described above. Similarly, the substrates for immobilization may be any of the common substrates, glass, plastic, a microarray, a microfluidics card, a chip or a chamber.

Labeled or Immobilized Ligands that Bind the Biomarkers or Peptides

In another embodiment, the diagnostic reagent is a ligand that binds to a biomarker recited above or a unique peptide thereof. Such a ligand desirably binds to a protein biomarker or a unique peptide contained therein, and can be an antibody which specifically binds a single biomarker described above, or a unique peptide in that single biomarker. Various forms of antibody, e.g., polyclonal, monoclonal, recombinant, chimeric, as well as fragments and components (e.g., CDRs, single chain variable regions, etc.) may be used in place of antibodies. The ligand itself may be labeled or immobilized.

In another aspect, suitable embodiments of such labeled or immobilized reagents include at least one, 2, 3, 4, 5, 6, 7, 8, 9, 10, 11, 12, 13, or 14 ligands. Each ligand binds to a single biomarker or their unique peptide fragments therein. In another aspect, other suitable embodiments of such labeled or immobilized reagents include an additional at least one, 2, or 3 ligands, wherein each ligand binds to a single biomarker or their unique peptide fragments therein.

Any combination of labeled or immobilized biomarker-binding ligands can be assembled in a diagnostic kit for the purposes of diagnosing EP. For example, one embodiment of a diagnostic kit includes labeled or immobilized reagents that bind to the groups of biomarkers identified in Table 1.

Labeled or Immobilized Polynucleotide/Oligonucleotides that Hybridize to Genes, Gene Fragments, Gene Transcripts of other Sequences Encoding the Biomarkers or Peptides

In another embodiment, the diagnostic reagent is a polynucleotide or oligonucleotide sequence that hybridizes to gene, gene fragment, gene transcript or nucleotide sequence encoding a biomarker of any one or more of the biomarkers described above or encoding a unique peptide thereof. Such a polynucleotide/oligonucleotide can be a probe or primer, and may itself be labeled or immobilized. In another aspect, suitable embodiments of such labeled or immobilized reagents include at least one, 2, 3, 4, 5, 6, 7, 8, 9, 10, 11, 12, 13, or 14 polynucleotide/oligonucleotide. Each polynucleotide/oligonucleotide hybridizes to a gene, gene fragment, gene transcript or expression product encoding a single biomarker or their unique peptide fragments therein. In another aspect, other suitable embodiments of such labeled or immobilized reagents include an additional at least one, 2, 3, 4 or 5 polynucleotide/oligonucleotides, wherein each sequence hybridizes to a gene, gene fragment, gene transcript of expression product encoding a single biomarker or their unique peptide fragments therein.

Any combination of labeled or immobilized biomarker-hybridizable sequences can be assembled in a diagnostic kit for the purposes of diagnosing EP. For example, one embodiment of a diagnostic kit includes labeled or immobilized reagents that hybridize to at least one of the biomarkers described above. Another embodiment of a diagnostic kit includes labeled or immobilized reagents that hybridize all of the biomarkers described above. Still other components of the many biomarker signatures that may be formed by various combinations of polynucleotide/oligonucleotide sequences that hybridize to the biomarkers described above, or their unique fragments associated with detectable labels or immobilized on substrates provide additional diagnostic kits. In one embodiment, these polynucleotide or oligonucleotide reagent(s) are part of a primer-probe set, and the kit comprises both primer and probe. Each said primer-probe set amplifies a different gene, gene fragment or gene expression product that encodes a different biomarker of any combination of the markers described above, optionally including one or more additional biomarkers. In still another embodiment, additional polynucleotide or oligonucleotide sequences in the diagnostic reagent or kit, hybridize to a gene, gene fragment, gene transcript or expression product identified in Table 1.

For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publicly available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, optimal PCR primers and probes used in the compositions described herein are generally 17-30 bases in length, and contain about 20-80%, such as, for example, about 50-60% G+C bases. Melting temperatures of between 5° and 80° C., e.g., about 50 to 70° C. are typically preferred.

Thus, a composition for diagnosing ectopic pregnancy or non-viable pregnancy in a mammalian subject as described herein can be a kit containing multiple reagents or one or more individual reagents. For example, one embodiment of a composition includes a substrate upon which the biomarkers, polynucleotides or oligonucleotides, or ligands are immobilized. In another embodiment, the composition is a kit also contains optional detectable labels, immobilization substrates, optional substrates for enzymatic labels, as well as other laboratory items.

The compositions based on the biomarkers selected from Table 1 or described herein, optionally associated with detectable labels, can be presented in the format of a microfluidics card, a chip or chamber, or a kit adapted for use with the assays described in the Examples, LC-MS/MS, ELISAs or PCR, RT-PCR or Q PCR techniques described herein.

The selection of the ligands, poly/oligonucleotide sequences, their length, suitable labels and substrates used in the composition are routine determinations made by one of skill in the art in view of the teachings of which biomarkers form signature suitable for the diagnosis of ectopic pregnancy.

### Methods of Determining Pregnancy Location and Viability

### Protein Assays

In one embodiment, a method for diagnosing an ectopic pregnancy in a female mammalian subject includes measuring in a biological fluid sample of the subject the expression level of a protein or peptide fragment thereof selected from at least one biomarker described above. Alternatively, the method includes measuring a combination of two or more biomarkers described above. The method further involves comparing the subject's expression level of the selected biomarker or biomarker fragment with the level of the same protein or peptide in the biological fluid of a reference or control female mammalian subject having a normal intrauterine pregnancy (IUP). Changes in expression of the subject's selected biomarker protein or peptide fragment from those of the reference or control correlates with a diagnosis of ectopic pregnancy.

In another embodiment, the above method further includes measuring in the biological fluid sample of the subject the expression level of an additional biomarker protein or peptide fragment. In another embodiment, the above method further includes measuring in the biological fluid sample of the subject the expression level of two or more additional biomarker protein or peptide fragments.

In this diagnostic method, a change in expression level of one or more of the selected biomarker proteins or peptide fragment in comparison to the IUP control reference may be an increase or decrease in the expression levels of the individual biomarkers. This method may employ any of the suitable diagnostic reagents or kits or compositions described above.

In certain embodiments, the measurement of the EP biomarkers in the samples employs an assay selected from mass spectrometry (MS), liquid chromatography (LC), liquid chromatography-mass spectrometry (LC-MS), targeted liquid chromatography-tandem mass spectrometry (LC-MS/MS), high-performance liquid chromatography (HPLC), ultra-performance liquid chromatography (UPLC), ultra-high-performance liquid chromatography (UHPLC), gas chromatography (GC), gas chromatography-mass spectrometry (GC-MS), globally optimized targeted mass spectrometry, targeted assay of about 200 metabolites, aqueous global profiling, liquid global profiling, GC-MS profiling, GC-MS flux analysis, carnitine analysis, lipid targeted analysis, quantitative lipid targeted analysis, tryptophan analysis, absolute quantification, multivariate statistical analysis, dynamic light scattering (DLS), nuclear magnetic resonance (NMR) spectroscopy, ultraviolet-visible (UV/Vis) spectroscopy, infrared (IR) spectroscopy, Raman spectroscopy, or any combination thereof.

In certain embodiments, the measurement of the EP biomarkers in the samples employs liquid chromatography-mass spectrometry/mass spectrometry (LC-MS/MS). LC-MS/MS combines the separating power of liquid chromatography with highly sensitive and selective mass analysis of mass spectrometry. In certain embodiments, a hybrid quadrupole-Orbitrap mas spectrometer is used for PRM analysis. The procedures for performing LC-MS/MS are well known to the skilled artisan (see e.g., US2019/0369116A1). Briefly, a solution containing the biomarkers is pumped through a LC column to separate out each biomarker. The sample then enters the mass spectrometer where the sample is ionized. By applying an electromagnetic field to the sample, the MS can quantify the biomarkers with a high degree of sensitivity and selectivity based on the unique mass/charge (m/z) transitions of each compound. This allows for a quantitative measurement of each biomarker in the solution.

In LC-MS/MS methods, one or more internal standard is used as a control to ensure that the sample has undergone the process, from preparation of the sample to completed analysis, in a correct manner. Any differences are compensated for by the added internal standard. It would therefore be advantageous to use one or more internal standards early in the method. This would allow for a better control of the quality of the analysis.

The LC-MS/MS contains at least one apparatus to perform liquid chromatography (LC). In one embodiment, the station for liquid chromatography comprises a column for extraction chromatography. Additionally or alternatively, the station for liquid chromatography comprises a column for analytical chromatography. In certain embodiments, the columns for extraction chromatography and analytical chromatography comprise a single station or single column. For example, in one embodiment, liquid chromatography is used to purify the biomarker of interest from other components in the sample that co-purify with the biomarker of interest after extraction or dilution of the sample. The LC-MS/MS also includes a tandem mass spectrometry station for analyzing the chromatographically separated one or more biomarkers of interest by mass spectrometry to determine the presence or amount of the one or more biomarkers in the test sample.

In other embodiments, the protein amount of each biomarker may be measured by any one method selected from the group consisting of protein mass analysis, protein chip analysis, an immune measurement method, a ligand binding assay, matrix desorption/ionization time of flight mass spectrometry (MALDI-TOF) analysis, surface enhanced laser desorption/ionization time of flight mass spectrometry (SELDI-TOF) analysis, a radioimmunoassay, a radial immunodiffusion method, an Ouchterlony immunodiffusion method, rocket immunoelectrophoresis, tissue immunostaining, a complement fixation assay method, 2-dimensional electrophoresis analysis, liquid chromatography-mass spectrometry (LC-MS), liquid chromatography-mass spectrometry/mass spectrometry (LC-MS/MS), western blot and enzyme linked immunosorbent assay (ELISA). The measurement of the EP biomarkers in the biological sample may employ any suitable ligand, e.g., antibody (or antibody to any second biomarker) to detect the EP biomarker protein. Such antibodies may be presently extant in the art or presently used commercially, such as those available as part of commercial antibody ELISA assay kits or that may be developed by techniques now common in the field of immunology. In certain embodiments, the biomarker reagents comprise one or more peptides, as identified in Table 5.

As used herein, the term “antibody” refers to an intact immunoglobulin having two light and two heavy chains or any fragments thereof. Thus, a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. A recombinant molecule bearing the binding portion of an EP biomarker antibody, e.g., carrying one or more variable chain CDR sequences that bind e.g., ISM2, may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same EP biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers or a particular sequence of the selected EP biomarkers may be employed in the methods of the present invention, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The EP biomarker-antibodies or fragments useful in this invention are not limited by the particular detectable label or label system employed. Thus, selection and/or generation of suitable EP biomarker antibodies with optional labels for use in this invention is within the skill of the art, provided with this specification, the documents incorporated herein, and the conventional teachings of immunology.

Similarly the particular assay format used to measure the selected EP biomarker in a biological sample may be selected from among a wide range of immunoassays, such as enzyme-linked immunoassays, such as those described in the examples below, sandwich immunoassays, homogeneous assays, immunohistochemistry formats, or other conventional assay formats. One of skill in the art may readily select from any number of conventional immunoassay formats to perform this invention.

Other reagents for the detection of protein in biological samples, such as peptide mimetics, synthetic chemical compounds capable of detecting the selected EP biomarker may be used in other assay formats for the quantitative detection of biomarker protein in biological samples, such as high pressure liquid chromatography (HPLC), immunohistochemistry, etc.

Employing ligand binding to the biomarker proteins or multiple biomarkers forming the signature enables more precise quantitative assays, as illustrated by the ELISA assays.

In one embodiment, suitable assays for use in these methods include immunoassays using antibodies or ligands to the above-identified biomarkers and biomarker signatures. In another embodiment, a suitable assay includes an ELISA assay for two more EP biomarkers that include one or more of the proteins/unique peptides described above. The platform most likely to be used in clinical assays will be multiplexed or parallel sandwich ELISA assays or their equivalent, primarily because this platform is the technology most commonly used to quantify blood proteins in clinical laboratories.

### Nucleic Acid Assays

Still other methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, CA), differential display, amplified fragment length polymorphism (iAFLP), and BeadArray™ technology (Illumina, San Diego, CA) using the commercially available Luminex100 LabMAP system and multiple color-coded microspheres (Luminex Corp., Austin, Tex.) and high coverage expression profiling (HiCEP) analysis.

Thus, in yet another embodiment, a method for diagnosing an ectopic pregnancy or a non-viable pregnancy in a female mammalian subject involves measuring in a biological fluid sample of the subject the expression level of a gene, gene fragment, gene transcript or expression product encoding one or more of the biomarkers. Alternatively, the method includes measuring the expression level of a gene, gene fragment, gene transcript or expression product encoding a combination of two or more biomarkers. The method further includes comparing the subject's selected biomarker gene, gene fragment, gene transcript or expression product expression level with the level of the same gene, gene fragment, gene transcript or expression product in the biological fluid of a reference or control female mammalian subject having a normal intrauterine pregnancy (IUP). Changes in expression of the subject's selected biomarker gene, gene fragment, gene transcript or expression products from those of the reference or control correlates with a diagnosis of ectopic pregnancy or non-viable pregnancy.

In another embodiment, the above method further includes measuring in the biological fluid sample of the subject the expression level of an additional biomarker gene, gene fragment, gene transcript or expression product encoding fragment of a biomarker. In another embodiment, the above method further includes measuring in the biological fluid sample of the subject the expression level of two or more additional biomarker gene, gene fragment, gene transcript or expression product encoding biomarkers.

In another embodiment, the above method further includes measuring in the biological fluid sample of the subject the expression level of an additional biomarker gene, gene fragment, gene transcript or expression product encoding fragment of a biomarker identified above.

In this diagnostic method, a change in expression level of one or more of the selected biomarker gene, gene fragment, gene transcript or expression product in comparison to the IUP control reference may be an upregulation or down regulation in the expression of the individual biomarkers gene, gene fragment, transcript or expression product. This method may employ any of the suitable diagnostic reagents or kits or compositions described above.

In yet another embodiment, the methods and compositions described herein may be used in conjunction with clinical risk factors to help physicians make more accurate decisions about how to manage patients with ectopic pregnancies. Another advantage of these methods and compositions is that diagnosis may occur early.

The following examples are illustrative only and are not intended to limit the present invention.

### EXAMPLES

### Example 1: Materials and Methods

### Plasma Collection and Processing

For the discovery (n=48) and validation (n=74) cohorts, plasma was collected from consenting women with early-stage pregnancies that were diagnosed either at the time of sample collection or shortly thereafter as IUP, EPL, or EP with each group having a similar distribution for gestational age (GA). Blood was collected by venipuncture into K2EDTA plasma tubes (BD, Franklin Lakes, NJ), and centrifuged for 10 min at 1,500×g at room temperature. Plasma was aliquoted in 500 μl volumes into cryotubes, snap frozen using liquid nitrogen within 2 h of blood collection, and stored at −80° C. Before downstream processing was performed, samples were thawed briefly in a RT water bath with intermittent periods of cooling on ice to prevent sample warming above 0-4° C. Thawed samples were centrifuged for 10 minutes at 12,000×g at 4° C., aliquoted into smaller volumes (40-100 μl), snap frozen using liquid nitrogen, and stored at −80° C. until used. Patient characteristics for samples are listed in Table 3.

TABLE 3
Demographic and clinical characteristics of the discovery (N = 48) and validation (N = 74) cohorts

| | Discovery Cohort | | | Validation Cohort | | |
|---|---|---|---|---|---|---|
| | IUP | EP | EPL | IUP | EP | EPL |
| | (N = 16) | (N = 16) | (N = 16) | (N = 25) | (N = 25) | (N = 24) |
| Race, n (%) | | | | | | |
| Black | 13 (81) | 15 (94) | 7 (44) | 15 (60) | 16 (64) | 12 (50) |
| White | 1 (6) | 1 (6) | 4 (25) | 3 (12) | 6 (24) | 7 (29) |
| Other | 2 (13) | 0 (0) | 0 (0) | 7 (28) | 3 (12) | 5 (21) |
| Unknown | 0 (0) | 0 (0) | 5 (31) | 0 (0) | 0 (0) | 0 (0) |
| Ethnicity, n (%) | | | | | | |
| Hispanic | 0 (0) | 0 (0) | 2 (12.5) | 5 (20) | 3 (12) | 1 (4) |
| Non-Hispanic | 14 (87.5) | 16 (100) | 12 (75) | 20 (80) | 21 (84) | 23 (96) |
| Unknown | 2 (12.5) | 0 (0) | 2 (12.5) | 0 (0) | 1 (4) | 0 (0) |
| Mean β-hCG (mIU/mL) | 29,983 | 8,706 | 24,892 | 47,150 | 5,924 | 21,177 |
| Unknown, n (%) | 0 (0) | 0 (0) | 0 (0) | 2 (8) | 2 (8) | 5 (21) |
| Mean GA based on last | | | | | | |
| menstrual period | | | | | | |
| Days | 45 | 48 | 63 | 54 | 46 | 71 |
| Unknown, n (%) | 0 (0) | 0 (0) | 1 (6) | 0 (0) | 0 (0) | 0 (0) |
| Mean GA based on | | | | | | |
| ultrasound | | | | | | |
| Days | 44 | NA | 49 | 49 | NA | 52 |
| Unknown, n (%) | 0 (0) | 0 (0) | 1 (6) | 2 (8) | 0 (0) | 3 (13) |

### IGY-14/Supermix Depletion

Samples were depleted of abundant plasma proteins using IGY-14 and Supermix immunodepletion columns (Sigma-Aldrich, St. Louis, MO) connected in tandem as previously described [10]. Typically, 100 μl (discovery cohort) or 50 μl (validation cohort) aliquots of plasma were thawed, centrifuged for 10 minutes at 12,000×g at 4° C., diluted five-fold with equilibration buffer, filtered through a 0.22 μm microcentrifuge filter, and injected onto the columns. The flow-through fractions containing unbound proteins were collected, pooled, and concentrated using a 10K MWCO centrifugal filter unit (MilliporeSigma, Burlington, MA), concentrator membranes were extracted with 1% SDS and extracts were combined with the concentrated sample. Concentrated samples were snap frozen, lyophilized using a SpeedVac centrifuge, and stored at −20° C. prior to 1-D SDS-PAGE and LC-MS/MS analysis.

### SDS-PAGE/In-Gel Trypsin Digestion

For plasma samples collected from the discovery cohort, IGY-14/Supermix-depleted and lyophilized samples were resuspended in SDS sample buffer, loaded onto pre-cast NUPAGE gels (Thermo Fisher Scientific, Waltham, MA), and separated using MES running buffer (Thermo Fisher Scientific) until the tracking dye had migrated 1.6 cm. Gels were stained with Colloidal Blue (Thermo Fisher Scientific), and the entire gel lane was excised and divided into six fractions, based on gel band staining, as previously described [11]. Fractions were digested overnight using 20 ng/ml modified trypsin. For plasma collected from the validation cohort, samples were processed similarly as described above with the exception that samples were run for 0.5 cm onto gels followed by overnight digestion using 10 ng/ml modified trypsin [11]. Digested samples were freeze-dried using a SpeedVac centrifuge and stored at −20° C. Dried samples were re-suspended in 0.1% formic acid/3% I or 3% ACN prior to LC-MS/MS discovery or PRM-MS validation, respectively.

### Stable Isotope Labeled (SIL) Peptide Standards Preparation

The “heavy” SIL peptides correspond to the heavy peptides in Table 5. These sequences were determined using LC-MS/MS spectrometry. Target peptides that could distinguish between highly homologous protein isoforms present in the blood were selected from the LC-MS/MS discovery analysis with consideration for common criteria of quantotypic peptides (e.g., length, tryptic specificity, modifications, and MS signal). Where possible, at least 2 peptides were selected per target protein.

Individual “heavy” SIL peptide stock solutions were prepared as follows: SpikeTides-TQL peptides (JPT Peptide Technologies, Berlin, Germany) were cleaved from their quantification tag (Qtag) prior to stock solution preparation [12]. Briefly, 1 nmol of each freeze-dried SIL peptide was solubilized in 25 mM ammonium bicarbonate/20% ACN and digested in-solution with 10 ng/μl trypsin (enzyme/peptide ratio of 1:100) in 25 mM ammonium bicarbonate overnight. Additionally, freeze-dried SpikeTides-L peptides (JPT Peptide Technologies) were resuspended and aliquoted in stock solutions of 10 pmol/μl in 10% ACN/2% formic acid, and AQUA peptides (Thermo Fisher Scientific) were aliquoted in stock solutions at 5 pmol/μl in 5% ACN.

Stock solutions of cleaved SpikeTides-TQL (18 total), SpikeTides-L (17 total), and AQUA peptides (2 total) were pooled at fmol amounts ranging from ~4 to 70 fmol each based on MS signal intensity that was pre-determined in quality control analyses of individual peptides. The pooled SIL peptide stock solution (10 pmol/μl) to be used for all subsequent quantitation analyses was aliquoted, snap frozen, and stored at −20° C. Prior to PRM-MS analysis, the pooled SIL peptide stock solution was thawed and diluted ten-fold to a final concentration of 1 pmol/μl in 0.1% formic acid/3% ACN/0.004% PEG. Next, 5 μl (5 pmol) was added to resuspended digests (35 μl) containing the equivalent of 15 μl of original plasma. PRM-MS sample injections (see below) contained the equivalent of 3 μl original plasma and 1 pmol of pooled SIL peptide standards.

### LC-MS/MS

Samples were analyzed on a Q Exactive HF mass spectrometer (Thermo Scientific) equipped with a nanoACQUITY ultrahigh pressure liquid chromatography (UPLC) System (Waters, Milford, MA) and a column heater maintained at 45° C. Tryptic digests were injected onto a UPLC Symmetry trap column (180 μm i.d.×2 cm packed with 5 μm C18 resin; Waters), and peptides were separated by reversed phase-ultra high pressure liquid chromatography (RP-UPLC) on a BEH C18 nanocapillary analytical column (75 μm i.d.×25 cm, 1.7 μm particle size, Waters) at a flow rate of 200 nl/min. Solvent A was Milli-Q (MilliporeSigma) water containing 0.1% formic acid, and solvent B was acetonitrile containing 0.1% formic acid. For the discovery cohort, peptides were eluted using a 70 min LC gradient as previously described [11].

### PRM-MS

For the validation cohort, samples were analyzed on a Q Exactive HF mass spectrometer (Thermo Scientific) equipped with a nanoACQUITY UPLC System (Waters, Milford, MA) as described above. Peptides were eluted at 200 nl/min using an acetonitrile gradient consisting of 5-30% B over 110 min, 30-40% B over 10 min, 40-80% B over 5 min, 80% B for 10 min before returning to 5% B over 0.5 min. The column was re-equilibrated using 5% B at 300 nl/min for 5 min before injecting the next sample. To minimize carryover, a blank was run between each experimental sample by injecting water and using a 30 min gradient with the same solvents. The PRM method consisted of a full MS scan (m/z 375-1150) acquired in profile mode at 30,000 resolution, followed by up to 20 MS/MS scans from an inclusion list containing the m/z, charge state, and retention time±5-6 min for each targeted peptide. PRM scans were acquired in profile mode at 30,000 resolution with a target AGC of 2×105 ions and max injection time of 120 ms. An isolation width of 0.7 m/z and normalized collision energy of 28% were used.

A reference plasma sample from a pool of all EPL plasma samples from the validation cohort was depleted, digested, and spiked with SIL peptide standards as described above, and then aliquots of the final digest with added internal standard SIL peptides were snap frozen. An aliquot of this reference sample was typically run at the beginning, middle, and end of each set of samples to monitor variations in PRM signal intensities caused by changes in performance of the HPLC, reversed-phase column, or mass spectrometer.

### MS Data Analysis

Raw mass spectrometric data from the proteomics discovery were processed using label-free quantitation (LFQ) with MaxQuant (v. 1.5.2.8) [13], using the “match between runs” option [14], as previously described [15]. Protein identifications were filtered using Perseus software (v. 1.6.2.3; available at perseus-framework.org) to remove decoy database reverse identifications, contaminants, proteins identified only by site modified peptides, or proteins identified by a single uniquely-mapping peptide.

In Perseus, protein group LFQ intensities were log 2 transformed to reduce the impact of outliers. For pairwise comparisons of the discovery analysis, samples were categorized into groups based on pregnancy outcome (EP, IUP, or EPL). Protein groups having less than 50% of valid values (i.e., those with MS1 quantification results) present in at least one categorical group were removed. Prior to statistical analysis, missing data points were imputed from a Gaussian distribution of random numbers that simulate the distribution of low signal values (distribution width=0.3, shift=1.8). Perseus was also used for data visualization using volcano plots.

For PRM-MS analyses of the validation cohort, raw data files were analyzed using Skyline (v. 21.2) [17], and automated fragment ion selection (5 ions/peptide) was utilized. The summed peak area of the 3-4 most intense fragment ions was used to quantify both “light” (i.e., endogenous) and “heavy” (i.e., SIL) peptides. Missing peaks and/or peptide fragment peaks with mass error >10 ppm were removed. For peptides containing methionine, both the oxidized and non-oxidized forms were quantified separately, and peak areas were summed prior to calculating abundance.

Calibration curves of individual SIL peptides were prepared using an EPL plasma pool as a background to evaluate matrix effects. To determine linear ranges, upper limits of quantitation (ULOQ), and lower limits of quantitation (LLOQs), a six-point dilution series of the SIL peptide pool (range: 64 fmol-2 pmol) was spiked into the EPL plasma pool and analyzed in duplicate by PRM-MS. Skyline was used to plot linear calibration curves and 1/×2 weighting was used. Peptides quantified in the 74 individual plasma samples that had quantities below the LLOQs were set to zero.

The abundance of each targeted peptide was calculated as the ratio between the light peptide and heavy peptide (L/H ratio). The amount of light peptide was calculated from the L/H ratio times the amount of heavy peptide spiked into the sample. Protein level in each sample was determined by taking the average of its targeted quantified peptides and the final protein concentration was calculated based on the volume of plasma analyzed.

### Statistical Methods

Statistical analyses were performed using Perseus software (v. 1.6.2.3), Microsoft Excel 2016, GraphPad Prism (v.5.04 and v.7), Stata 16, and R (v.4.2.1). For the discovery cohort, samples were grouped to identify differences related to early pregnancy complications such as EP or EPL. For the pairwise comparisons, two-tailed, two-sample Student's t-test statistic was calculated, and a permutation-based false discovery rate (FDR) was applied (FDR≤0.05, 250 permutations, S0=0.1) [18]. High priority (FDR≤0.05) and additional significant (p≤0.05 and fold change ≥3) candidate biomarkers were selected for further comparison between EP vs non-EP by a non-parametric Wilcoxon rank-sum test. Additionally, area under the curve (AUC) from receiver operating characteristic (ROC) curves were assessed (Table 4).

TABLE 4
Comparisons of AUC between candidate EP vs non-EP biomarkers
identified in the proteomics discovery cohort (N = 48)

| | | | | | 95% | | |
|---|---|---|---|---|---|---|---|
| | | | | Std. | confidence | | P |
| Gene name | Protein Description | N | AUC | Error | interval | | value |
| PAEP | Glycodelin | 48 | 0.898 | 0.046 | 0.808 | 0.989 | <0.0001 |
| PSG9 | Pregnancy-specific beta-1-glycoprotein 9 | 48 | 0.785 | 0.077 | 0.633 | 0.937 | 0.001 |
| PSG3 | Pregnancy-specific beta-1-glycoprotein 3 | 48 | 0.783 | 0.083 | 0.620 | 0.946 | 0.002 |
| CGB | Choriogonadotropin subunit beta | 48 | 0.779 | 0.083 | 0.617 | 0.942 | 0.002 |
| ISM2 | Isthmin-2 | 48 | 0.775 | 0.080 | 0.619 | 0.932 | 0.002 |
| PSG2 | Pregnancy-specific beta-1-glycoprotein 2 | 48 | 0.774 | 0.073 | 0.632 | 0.917 | 0.002 |
| CGA | Glycoprotein hormones alpha chain | 48 | 0.766 | 0.084 | 0.600 | 0.931 | 0.003 |
| PSG1 | Pregnancy-specific beta-1-glycoprotein 1 | 48 | 0.764 | 0.083 | 0.602 | 0.926 | 0.003 |
| NOTUM | Protein notum homolog | 48 | 0.703 | 0.084 | 0.539 | 0.867 | 0.023 |
| PAPPA | Pappalysin-1 | 48 | 0.681 | 0.088 | 0.509 | 0.852 | 0.043 |
| PSG11 | Pregnancy-specific beta-1-glycoprotein 11 | 48 | 0.666 | 0.088 | 0.494 | 0.838 | 0.063 |
| ADAM12 | Disintegrin and metalloproteinase domain- | 48 | 0.664 | 0.084 | 0.499 | 0.829 | 0.066 |
| | containing protein 12 | | | | | | |

To predict EP in the validation cohort, each biomarker was assessed using Wilcoxon rank-sum test with FDR adjustment calculated using Benjamini-Hochberg correction. Those markers with FDR≤0.05 were further evaluated as potential predictors with least absolute shrinkage and selection operator (Lasso) regularization and logistic regression being used to explore biomarkers that may be used for better prediction of EP than models with a single predictor. For Lasso and logistical regression analyses, zero values were set as 0.01 and then the protein concentrations for all candidate biomarkers were log 2 transformed. Correlations between candidate biomarkers were examined using Spearman's rank correlation coefficient. Final predictors for the multivariable logistic model were selected using the Lasso technique with the 5-fold cross-validation and one-standard-error rule for determining the optimal tuning parameter. Due to the modest sample size of the study, variables selection was determined using 100 independent rounds runs of 5-fold cross-validation Lasso. The biomarkers that were selected 80 or more times from 100 runs were used as a final set of predictors in logistic model. Additionally, three other models were explored using protein substitutions based on the Spearman correlation cluster analysis. The predictive ability of the final logistic models was assessed by AUC, sensitivity, and specificity.

### Example 2: Identification and Validation of Biomarkers

### Results

### Identification of Candidate Biomarkers in a Discovery Cohort

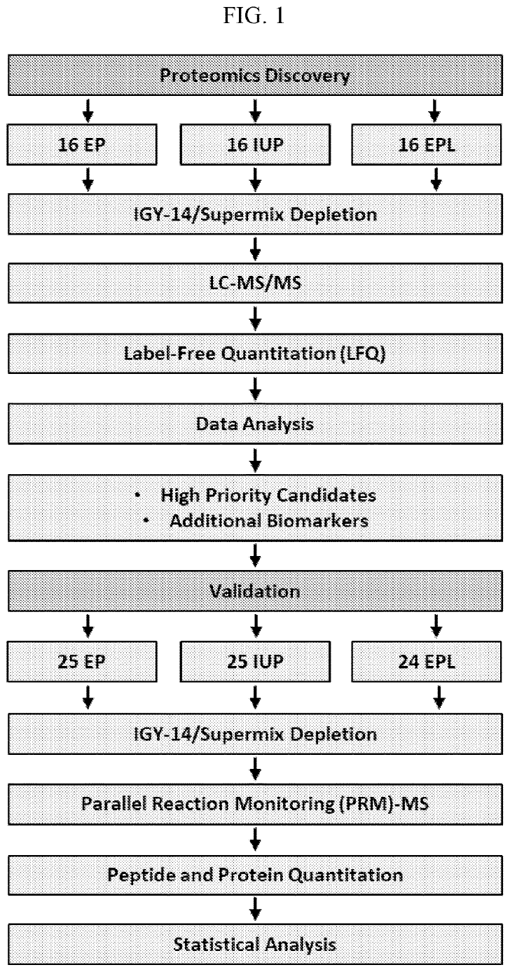

The scheme for label-free proteomics discovery of abundant-protein-depleted plasma samples from a cohort of 48 pregnant women with IUP, EP, and EPL is shown in FIG. 1.

This analysis initially identified ~2200 proteins by 2 or more peptides. During preliminary data evaluation, we noticed a correlation between abundance levels of some proteins and collection date across clinical conditions. To avoid potential plasma storage bias, we systematically evaluated protein levels vs total months the plasma was stored at −80° C. across all samples and filtered out proteins that showed decreased protein levels with increased length of storage (i.e., Pearson correlation with storage >−0.25). The remaining 1391 “storage stable” proteins were compared across clinical conditions.

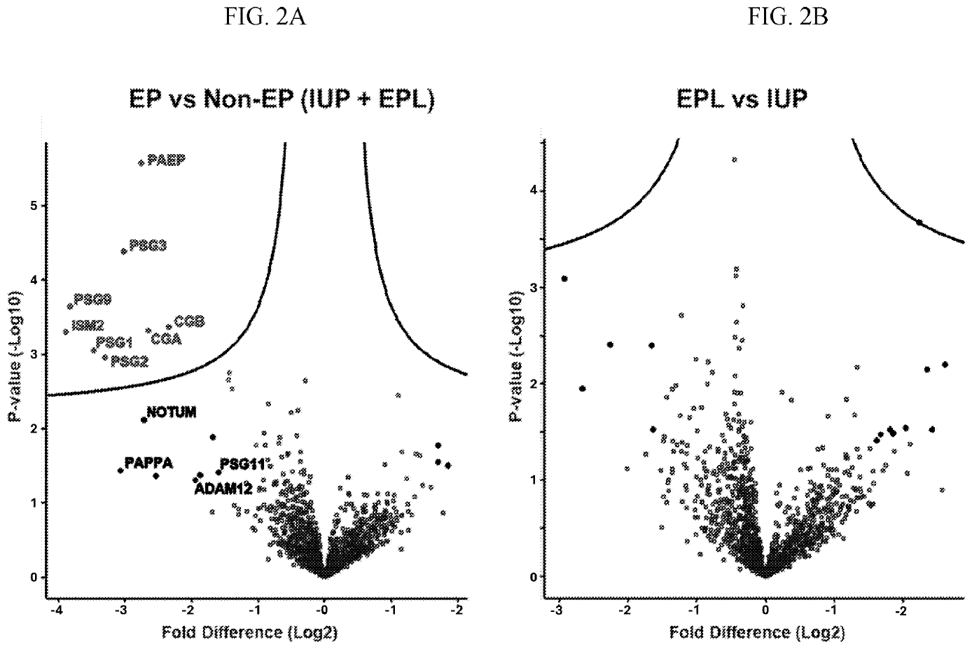

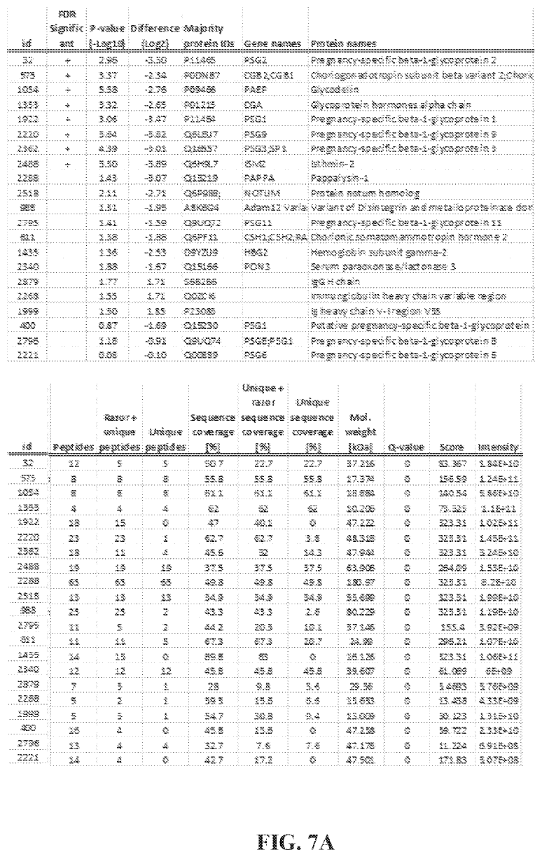

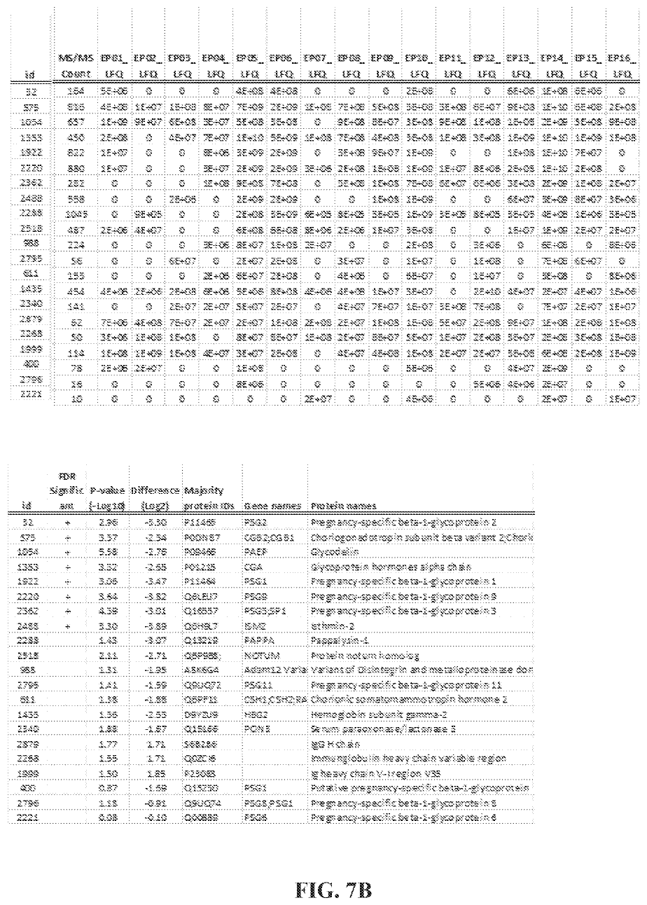

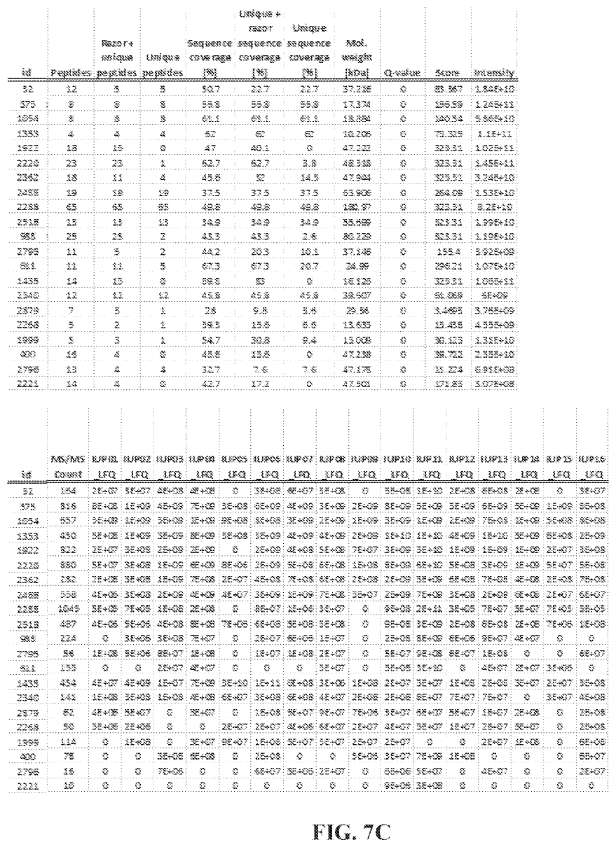

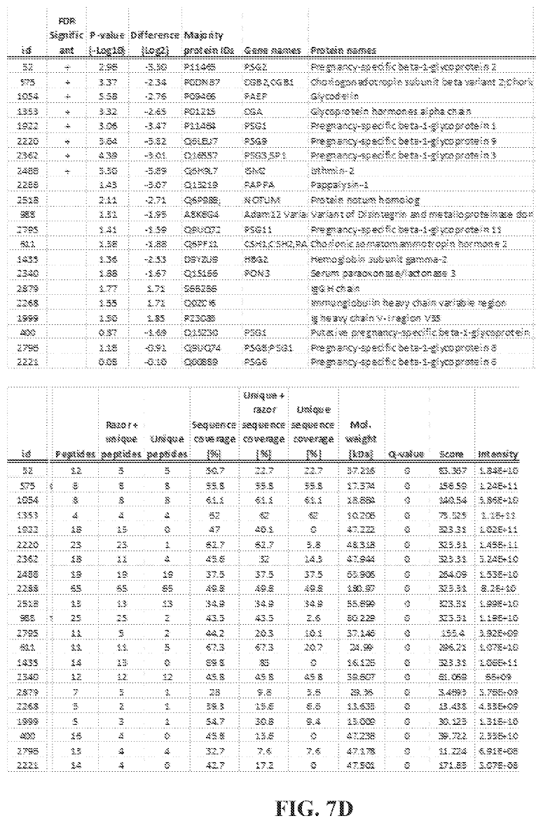

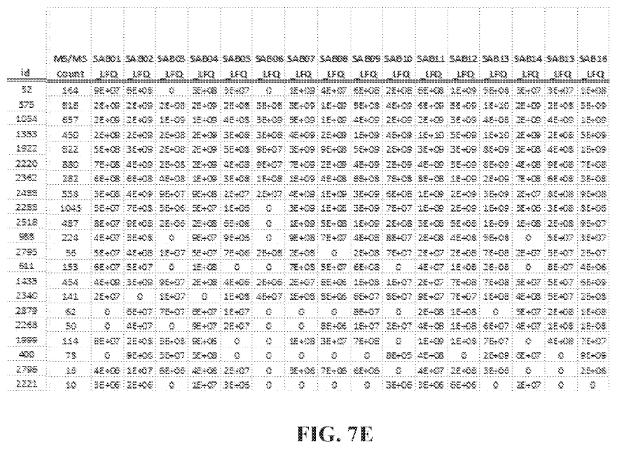

To identify potential biomarkers for the different clinical conditions, we performed all possible pairwise comparisons between the three groups as well as comparison of EP vs non-EP (IUP and EPL). The most promising biomarkers were identified from the comparison of EP vs non-EP, which yielded eight high priority candidates (FDR≤0.05), including CGB, glycoprotein hormones alpha chain (CGA), isthmin-2 (ISM2), glycodelin (PAEP), pregnancy-specific beta-1-glycoprotein 1 (PSG1), pregnancy-specific beta-1-glycoprotein 2 (PSG2), pregnancy-specific beta-1-glycoprotein 3 (PSG3), and pregnancy-specific beta-1-glycoprotein 9 (PSG9) (FIG. 2A, FIG. 7).

Interestingly, no high priority biomarkers were identified for EPL vs IUP (FIG. 2B). In order to cast a relatively “wide net” for subsequent EP biomarker validation, we also considered several additional candidate biomarkers that did not pass the FDR cutoff but were significant based on Student's t-test p-value≤0.05 and fold change ≥3, including disintegrin and metalloproteinase domain-containing protein 12 (ADAM12), palmitoleoyl-protein carboxylesterase NOTUM (NOTUM), pappalysin-1 (PAPPA), and pregnancy-specific beta-1-glycoprotein 11 (PSG11) (FIG. 2A, Table 4). Several other protein candidates meeting these relaxed criteria were also initially evaluated but subsequently dropped due to lack of reproducibly quantifiable, isoform-specific peptides as determined in preliminary PRM-MS assays.

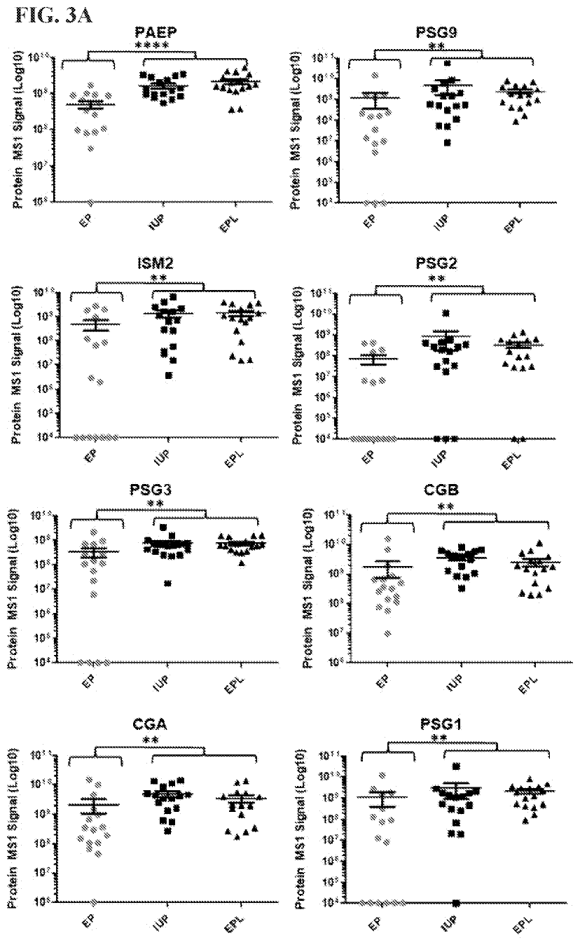

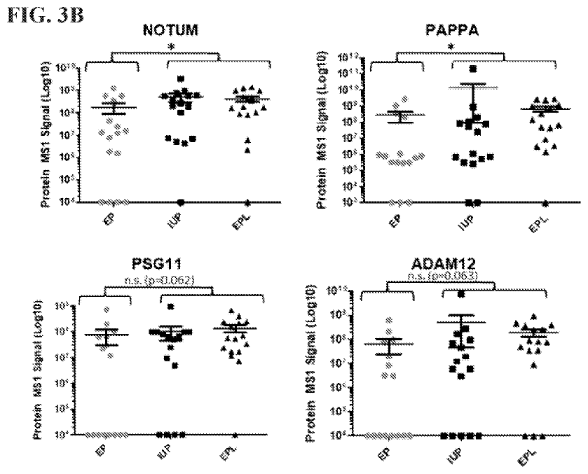

We also did not pursue several abundant blood proteins because their residual abundance levels were likely to be affected by variable recoveries from the major protein immunoaffinity depletion step. Scatter plots for the eight high priority and four additional significant candidate biomarkers across pregnancy outcomes are shown in FIG. 3. For each protein, the levels for EP samples were lower than for IUP and EPL samples. These results were consistent with previous biomarker discovery analysis of serum pools from several EP and IUP patients where a general characteristic was that all candidate EP biomarkers were lower abundance in EP compared to IUP [10]. Importantly, in the current, more in-depth proteome analysis with larger cohorts, the 12 candidate EP biomarkers exhibited good diagnostic accuracy with AUC≥0.664 for EP vs non-EP (Table 3).

### Assessment of PRM-MS Assay Performance

We next developed a multiplexed quantitative PRM-MS assay that utilized SIL internal standard peptides for absolute quantitation of the 12 candidate EP biomarkers. An important feature in selecting targeted peptides was to ensure that the peptides could distinguish between highly homologous protein isoforms present in the blood including the very complex PSG protein family. We evaluated the robustness of the assay using multiple strategies based on the 36 monitored peptides selected for our candidate biomarkers (Table 5).

TABLE 5
Biomarker peptide summary

| | | | | | | | |
|---|---|---|---|---|---|---|---|
| | Light | Heavy | | Reference | | | |
| Gene Name/Peptide Sequence | Precursor | Precursor | Precursor | Ratio | fmol on | LLOQ | ULOQ |
| (SEQ ID NO) | m/z | m/z | Charge | % CV | Column | (fmol) | (fmol) |

ADAM12

| | | | | | | | |
|---|---|---|---|---|---|---|---|
| NHPEVLNIR (1) | 546.30 | 551.31 | 2 | 14.04 | 6 | 0.0192 | 60 |
| VNSAGDPYGNCGK (2) | 669.79 | 673.80 | 2 | 14.71 | 16 | 0.0512 | 32 |
| FGFGGSTDSGPIR (3) | 649.31 | 654.32 | 2 | 21.60 | 35 | 0.0224 | 350 |

ISM2

| | | | | | | | |
|---|---|---|---|---|---|---|---|
| TPEVTPLR (4) | 456.76 | 461.77 | 2 | 15.61 | 5 | 0.08 | 50 |
| TRPCGYGCTATETR (5) | 543.91 | 547.24 | 3 | 5.69 | 20 | 0.064 | 200 |
| LDIYQPTAR (6) | 538.79 | 543.79 | 2 | 8.42 | 4 | 0.00256 | 40 |
| FDTTPWILCK (7) | 640.82 | 644.83 | 2 | 16.74 | 5 | 0.016 | 50 |
| ACTDNPLEEEYLAQLQEAK (8) | 1111.52 | 1115.52 | 2 | 13.93 | 5 | 0.08 | 10 |

PAEP

| | | | | | | | |
|---|---|---|---|---|---|---|---|
| VHITSLLPTPEDNLEIVLHR (9) | 766.09 | 769.43 | 3 | 4.63 | 15 | 1.2 | 30 |
| HLWYLLDLK (10) | 600.84 | 604.85 | 2 | 8.21 | 25 | 0.4 | 50 |
| VLVEDDEIMQGFIR_SUM (11) | 832.42 | 837.43 | 2 | 6.58 | 46 | 0.736 | 460 |

NOTUM

| | | | | | | | |
|---|---|---|---|---|---|---|---|
| LGYPAIQVR (12) | 508.80 | 513.80 | 2 | 24.63 | 11 | 0.00704 | 22 |
| DVPASFAPACLSHEIIIR (13) | 666.02 | 669.35 | 3 | 17.32 | 20 | 0.064 | 40 |
| SHWTDVQVK (14) | 550.28 | 554.29 | 2 | 33.70 | 19 | 0.0608 | 190 |

CGA

| | | | | | | | |
|---|---|---|---|---|---|---|---|
| AYPTPLR (15) | 409.23 | 414.24 | 2 | 12.30 | 14 | 0.224 | 28 |
| VTVMGGFK SUM (16) | 419.73 | 423.74 | 2 | 16.29 | 34 | 0.544 | 340 |

CGB

| | | | | | | | |
|---|---|---|---|---|---|---|---|
| VLQGVLPALPQVVCNYR (17) | 963.54 | 968.54 | 2 | 6.93 | 25 | 0.016 | 50 |
| DHPLTCDDPR (18) | 409.18 | 412.52 | 3 | 11.72 | 49 | 0.784 | 98 |

PAPPA

| | | | | | | | |
|---|---|---|---|---|---|---|---|
| SPAVITGLYDK (19) | 582.32 | 586.33 | 2 | 13.23 | 4 | 0.064 | 40 |
| AYLDVNELK (20) | 532.78 | 536.79 | 2 | 25.06 | 6 | 0.048 | 60 |

PSG1

| | | | | | | | |
|---|---|---|---|---|---|---|---|
| FQLPGQK (21) | 409.23 | 413.24 | 2 | 4.97 | 20 | 0.0128 | 200 |

PSG2

| | | | | | | | |
|---|---|---|---|---|---|---|---|
| IHPSYTNYR (22) | 575.79 | 580.79 | 2 | 17.54 | 10 | 0.032 | 100 |
| FQQSGQNLFIPQITTK (23) | 925.49 | 929.50 | 2 | 12.82 | 12 | 0.00768 | 120 |
| NSATGEESSTSLTVK (24) | 755.87 | 759.87 | 2 | 22.62 | 19 | 0.01216 | 190 |
| IGLLPLLNPT (25) | 525.83 | 529.34 | 2 | 11.23 | 22 | 0.352 | 220 |

PSG3

| | | | | | | | |
|---|---|---|---|---|---|---|---|
| EDAGSYTLHIVK (26) | 444.90 | 447.57 | 3 | 13.24 | 6 | 0.096 | 12 |
| VSAPSGTGHLPGLNPL(27) | 758.91 | 761.92 | 2 | 32.12 | 4 | 0.064 | 40 |

PSG6/9

| | | | | | | | |
|---|---|---|---|---|---|---|---|
| TLYLFGVTK (28) | 521.30 | 525.31 | 2 | 32.43 | 10 | 0.032 | 20 |

PSG8/1

| | | | | | | | |
|---|---|---|---|---|---|---|---|
| TLFLLGVTK (29) | 496.31 | 500.32 | 2 | 29.89 | 20 | 0.064 | 200 |

PSG9

| | | | | | | | |
|---|---|---|---|---|---|---|---|
| LPIPYITINNLNPR (30) | 819.47 | 824.48 | 2 | 14.69 | 69 | 0.2208 | 138 |
| SNPVILNVLYGPDLPR (31) | 883.99 | 889.00 | 2 | 22.33 | 15 | 0.0096 | 30 |
| LFIPQITR (32) | 494.30 | 499.31 | 2 | 12.67 | 5 | 0.0032 | 50 |
| VSGPCHGDLTESQS (33) | 737.32 | 740.32 | 2 | 13.02 | 40 | 0.0128 | 400 |

PSG11

| | | | | | | | |
|---|---|---|---|---|---|---|---|
| YTAGPYECEIWNSGSASR (34) | 1024.44 | 1029.45 | 2 | 8.85 | 12 | 0.192 | 24 |
| LFIPQITPK (35) | 528.83 | 532.83 | 2 | 31.19 | 14 | 0.00896 | 140 |
| VIAPPGLGTFAFNNPT (36) | 808.43 | 811.43 | 2 | 11.71 | 17 | 0.272 | 170 |

First, we determined the linearity, ULOQ, and LLOQ for each targeted peptide by producing a standard curve in a major protein depleted plasma background (Table 5). The amounts of each peptide detected in each of the 74 individual plasma samples were compared to these limits, and values outside the range were flagged. All but one peptide, which was subsequently dropped, yielded quantifiable values for at least 90% of samples within at least one patient group.

We also prepared a reference plasma sample from a pool of EPL plasma samples. Because the 74 individual plasma samples had to be processed and analyzed in multiple batches, this reference was used throughout the analyses to monitor consistency of quantitation both within an experiment and between batches by analyzing this sample at the beginning and end of each batch of LC-MS/MS runs when feasible. We observed excellent consistency of quantitation of the reference plasma sample across the entire experiment. The coefficients of variation (CVs) for most SIL peptides were <25% within a series of LC-MS/MS runs and all peptides had CVs <35% (Table 5).

### Validation of EP Versus Non-EP Candidate Biomarkers in an Independent Cohort.

We assessed the potential clinical utility of individual biomarkers by determining the ability of each biomarker to distinguish EP vs non-EP in an independent cohort of 74 patients. Protein concentrations (ng/ml) for each of the markers in the 74 patient plasma samples are listed in Table 6.

TABLE 6
Protein concentrations of biomarkers in the validation cohort

| Gene | EP01 | EP02 | EP03 | EP04 | EP05 | EP06 | EP07 | EP08 |
|---|---|---|---|---|---|---|---|---|
| Name | ng/mL | ng/mL | ng/mL | ng/mL | ng/mL | ng/mL | ng/mL | ng/mL |
| ADAM12 | 0.39 | 4.25 | 1.01 | 29.59 | 1.83 | 0.43 | 0.28 | 1.37 |
| ISM2 | 0.21 | 2.96 | 1.63 | 6.62 | 0.12 | 0.18 | 0.04 | 0.24 |
| PAEP | 44.53 | 11.72 | 45.05 | 28.45 | 26.05 | 12.47 | 48.42 | 16.39 |
| NOTUM | 1.61 | 6.74 | 3.98 | 18.13 | 0.49 | 1.80 | 0.97 | 2.48 |
| CGA | 37.72 | 87.71 | 53.04 | 373.06 | 52.89 | 71.11 | 6.87 | 11.66 |
| CGB | 55.18 | 122.83 | 73.73 | 509.35 | 78.85 | 133.54 | 16.75 | 25.75 |
| PAPPA | 0.00 | 0.00 | 0.00 | 6.18 | 0.00 | 0.00 | 0.00 | 0.00 |
| PSG1 | 2.23 | 8.43 | 3.08 | 27.00 | 3.56 | 2.27 | 0.44 | 2.83 |
| PSG2 | 0.09 | 1.55 | 0.56 | 9.28 | 0.45 | 0.58 | 0.00 | 0.49 |
| PSG3 | 6.78 | 18.16 | 9.00 | 19.70 | 2.67 | 4.61 | 0.00 | 6.23 |
| PSG6/9 | 1.97 | 5.51 | 1.53 | 32.81 | 0.00 | 3.29 | 0.00 | 2.73 |
| PSG8/1 | 10.05 | 36.28 | 14.53 | 101.73 | 14.50 | 10.81 | 1.94 | 15.58 |
| PSG9 | 0.44 | 2.06 | 0.26 | 12.49 | 0.10 | 0.82 | 0.03 | 0.25 |
| PSG11 | 0.33 | 0.40 | 0.23 | 6.73 | 0.10 | 0.00 | 0.00 | 0.66 |

| Gene | EP09 | EP10 | EP11 | EP12 | EP13 | EP14 | EP15 |
|---|---|---|---|---|---|---|---|
| Name | ng/mL | ng/mL | ng/mL | ng/mL | ng/mL | ng/mL | ng/mL |
| ADAM12 | 0.36 | 4.07 | 6.11 | 0.36 | 3.36 | 22.45 | 0.37 |
| ISM2 | 0.17 | 6.43 | 0.82 | 0.20 | 1.51 | 4.78 | 0.62 |
| PAEP | 33.29 | 5.25 | 37.59 | 4.60 | 163.61 | 58.50 | 89.54 |
| NOTUM | 1.14 | 10.29 | 3.00 | 1.59 | 4.11 | 8.69 | 2.02 |
| CGA | 21.71 | 141.48 | 41.37 | 8.78 | 61.26 | 115.35 | 22.09 |
| CGB | 42.58 | 279.01 | 64.82 | 18.81 | 111.45 | 185.70 | 52.82 |
| PAPPA | 0.00 | 0.00 | 0.00 | 0.00 | 0.00 | 14.18 | 0.00 |
| PSG1 | 1.67 | 11.01 | 7.71 | 1.42 | 6.07 | 34.80 | 1.26 |
| PSG2 | 0.39 | 2.33 | 0.78 | 0.15 | 0.49 | 9.86 | 0.20 |
| PSG3 | 4.34 | 10.99 | 5.22 | 2.56 | 10.91 | 13.06 | 3.25 |
| PSG6/9 | 2.77 | 22.56 | 6.03 | 2.46 | 8.28 | 39.61 | 1.38 |
| PSG8/1 | 7.63 | 52.44 | 32.91 | 7.03 | 25.06 | 125.29 | 6.01 |
| PSG9 | 0.70 | 12.83 | 2.45 | 0.86 | 2.30 | 24.87 | 0.18 |
| PSG11 | 0.05 | 1.78 | 0.20 | 0.40 | 2.32 | 7.67 | 0.16 |

| Gene | EP16 | EP17 | EP18 | EP19 | EP20 | EP21 | EP22 | EP23 | EP24 | EP25 |
|---|---|---|---|---|---|---|---|---|---|---|
| Name | ng/mL | ng/mL | ng/mL | ng/mL | ng/mL | ng/mL | ng/mL | ng/mL | ng/mL | ng/mL |
| ADAM12 | 0.79 | 1.00 | 0.84 | 1.21 | 1.21 | 0.45 | 10.15 | 25.83 | 6.94 | 2.34 |
| ISM2 | 0.13 | 0.34 | 0.10 | 0.10 | 0.15 | 0.07 | 1.94 | 1.31 | 2.41 | 0.20 |
| PAEP | 53.64 | 17.53 | 21.23 | 62.21 | 2.80 | 272.02 | 12.49 | 1.03 | 23.46 | 2.68 |
| NOTUM | 2.14 | 2.41 | 1.36 | 0.86 | 1.32 | 0.73 | 11.11 | 6.08 | 5.45 | 1.87 |
| CGA | 8.56 | 1.43 | 7.26 | 4.64 | 12.59 | 12.83 | 167.32 | 34.59 | 211.13 | 56.96 |
| CGB | 8.65 | 1.04 | 7.81 | 2.68 | 10.96 | 13.05 | 214.60 | 33.04 | 185.00 | 68.43 |
| PAPPA | 0.00 | 0.00 | 0.00 | 0.00 | 0.00 | 0.00 | 9.55 | 35.20 | 3.41 | 0.00 |
| PSG1 | 2.20 | 7.58 | 1.48 | 2.55 | 4.25 | 1.45 | 51.13 | 32.00 | 18.19 | 6.58 |
| PSG2 | 0.59 | 1.28 | 0.44 | 0.73 | 0.77 | 0.43 | 1.14 | 3.51 | 5.29 | 0.58 |
| PSG3 | 4.25 | 12.73 | 2.84 | 1.66 | 8.04 | 2.52 | 18.10 | 9.77 | 13.60 | 7.55 |
| PSG6/9 | 3.33 | 13.84 | 2.26 | 4.33 | 7.53 | 1.98 | 47.65 | 24.40 | 54.25 | 10.43 |
| PSG8/1 | 9.32 | 42.20 | 6.14 | 11.13 | 19.27 | 3.53 | 208.23 | 128.89 | 78.01 | 31.80 |
| PSG9 | 1.07 | 3.59 | 0.34 | 0.59 | 2.37 | 0.59 | 15.44 | 6.72 | 13.88 | 1.16 |
| PSG11 | 0.68 | 1.33 | 0.00 | 0.42 | 0.68 | 0.40 | 2.76 | 1.01 | 3.42 | 0.51 |

| Gene | IUP01 | IUP02 | IUP03 | IUP04 | IUP05 | IUP06 | IUP07 | IUP08 |
|---|---|---|---|---|---|---|---|---|
| Name | ng/mL | ng/mL | ng/mL | ng/mL | ng/mL | ng/mL | ng/mL | ng/mL |
| ADAM12 | 49.65 | 25.16 | 22.49 | 158.41 | 3.36 | 107.87 | 31.13 | 125.93 |
| ISM2 | 11.06 | 11.80 | 14.41 | 26.46 | 3.26 | 18.98 | 11.16 | 45.10 |
| PAEP | 166.88 | 70.35 | 200.38 | 116.86 | 35.96 | 115.90 | 112.98 | 130.35 |
| NOTUM | 22.33 | 24.84 | 30.74 | 75.77 | 8.18 | 29.24 | 14.35 | 62.54 |
| CGA | 707.58 | 601.85 | 431.25 | 1868.98 | 140.84 | 610.74 | 564.75 | 557.02 |
| CGB | 775.13 | 674.25 | 521.44 | 2074.76 | 235.53 | 1017.81 | 823.72 | 1126.99 |
| PAPPA | 21.67 | 10.77 | 13.41 | 102.74 | 0.00 | 45.69 | 14.41 | 199.42 |
| PSG1 | 50.28 | 113.72 | 59.38 | 324.70 | 13.56 | 254.28 | 70.83 | 298.87 |
| PSG2 | 16.30 | 24.26 | 7.43 | 74.91 | 2.96 | 17.16 | 13.76 | 68.93 |
| PSG3 | 24.91 | 19.86 | 16.06 | 21.88 | 8.13 | 17.72 | 20.91 | 17.01 |
| PSG6/9 | 46.85 | 92.22 | 39.17 | 403.36 | 10.20 | 250.57 | 45.66 | 338.33 |
| PSG8/1 | 222.54 | 365.25 | 189.85 | 1185.47 | 57.43 | 934.73 | 287.19 | 1051.63 |
| PSG9 | 12.90 | 44.52 | 13.58 | 176.17 | 2.01 | 156.92 | 17.77 | 101.62 |
| PSG11 | 6.43 | 17.59 | 0.51 | 53.19 | 1.68 | 52.22 | 12.39 | 56.78 |

| Gene | IUP09 | IUP10 | IUP11 | IUP12 | IUP13 | IUP14 | IUP15 |
|---|---|---|---|---|---|---|---|
| Name | ng/mL | ng/mL | ng/mL | ng/mL | ng/mL | ng/mL | ng/mL |
| ADAM12 | 43.56 | 319.41 | 8.66 | 9.18 | 52.15 | 23.57 | 5.20 |
| ISM2 | 11.31 | 37.79 | 8.94 | 4.51 | 7.09 | 13.30 | 11.09 |
| PAEP | 116.41 | 151.61 | 121.82 | 126.73 | 179.63 | 126.37 | 152.63 |
| NOTUM | 17.41 | 59.56 | 11.80 | 8.09 | 12.04 | 16.49 | 14.07 |
| CGA | 492.29 | 747.85 | 259.16 | 177.70 | 424.70 | 526.25 | 328.84 |
| CGB | 750.95 | 1155.82 | 379.06 | 366.98 | 780.39 | 976.72 | 544.52 |
| PAPPA | 5.16 | 124.01 | 6.96 | 3.57 | 12.23 | 14.08 | 2.12 |
| PSG1 | 74.45 | 422.13 | 29.45 | 24.51 | 82.07 | 59.88 | 10.79 |
| PSG2 | 8.37 | 49.59 | 2.76 | 6.11 | 6.24 | 19.82 | 1.58 |
| PSG3 | 14.34 | 21.48 | 12.20 | 12.60 | 14.45 | 16.46 | 16.51 |
| PSG6/9 | 69.31 | 347.86 | 23.18 | 22.46 | 60.04 | 130.85 | 23.39 |
| PSG8/1 | 258.06 | 1346.51 | 105.39 | 96.67 | 295.64 | 209.15 | 42.48 |
| PSG9 | 44.83 | 164.73 | 10.51 | 10.15 | 29.39 | 78.81 | 12.45 |
| PSG11 | 21.76 | 35.98 | 7.22 | 2.17 | 13.58 | 4.15 | 0.39 |

| Gene | IUP16 | IUP17 | IUP18 | IUP19 | IUP20 | IUP21 | IUP22 | IUP23 | IUP24 | IUP25 |
|---|---|---|---|---|---|---|---|---|---|---|
| Name | ng/mL | ng/mL | ng/mL | ng/mL | ng/mL | ng/mL | ng/mL | ng/mL | ng/mL | ng/mL |
| ADAM12 | 15.35 | 11.83 | 24.67 | 8.97 | 16.42 | 17.24 | 13.94 | 9.53 | 4.30 | 52.57 |
| ISM2 | 10.36 | 5.10 | 9.35 | 3.82 | 6.54 | 6.76 | 5.18 | 4.06 | 1.88 | 7.23 |
| PAEP | 124.62 | 167.41 | 223.95 | 173.33 | 103.04 | 62.59 | 227.02 | 107.68 | 102.46 | 195.65 |
| NOTUM | 32.16 | 17.70 | 29.76 | 16.56 | 24.08 | 17.10 | 24.65 | 8.45 | 7.48 | 16.92 |
| CGA | 78.35 | 364.06 | 164.40 | 118.96 | 179.35 | 179.00 | 243.00 | 339.84 | 196.95 | 648.99 |
| CGB | 72.53 | 320.73 | 150.25 | 120.06 | 175.39 | 160.34 | 243.44 | 260.42 | 169.29 | 514.16 |
| PAPPA | 2.33 | 8.25 | 14.51 | 0.00 | 5.12 | 10.75 | 9.49 | 6.28 | 0.00 | 17.55 |
| PSG1 | 53.93 | 29.01 | 80.81 | 34.17 | 68.00 | 35.31 | 46.48 | 83.76 | 10.82 | 173.26 |
| PSG2 | 16.24 | 5.41 | 8.40 | 4.85 | 9.57 | 12.63 | 10.30 | 11.25 | 1.28 | 26.40 |
| PSG3 | 12.83 | 15.32 | 20.06 | 12.06 | 17.83 | 16.36 | 20.65 | 14.22 | 21.90 | 18.59 |
| PSG6/9 | 36.01 | 39.43 | 89.11 | 34.47 | 99.89 | 54.35 | 27.46 | 70.13 | 8.13 | 122.26 |
| PSG8/1 | 215.92 | 127.26 | 317.17 | 126.60 | 194.20 | 143.10 | 191.40 | 319.30 | 58.32 | 611.39 |
| PSG9 | 6.64 | 9.77 | 29.89 | 12.40 | 25.80 | 9.55 | 9.01 | 15.13 | 0.50 | 41.50 |
| PSG11 | 7.94 | 5.52 | 5.71 | 2.55 | 1.71 | 5.24 | 6.67 | 3.92 | 0.86 | 28.54 |

| Gene | SAB01 | SAB02 | SAB03 | SAB04 | SAB06 | SAB07 | SAB08 | SAB09 | SAB10 | SAB11 | SAB12 | SAB13 | SAB14 | SAB15 |
|---|---|---|---|---|---|---|---|---|---|---|---|---|---|---|
| Name | ng/mL | ng/mL | ng/mL | ng/mL | ng/mL | ng/mL | ng/mL | ng/mL | ng/mL | ng/mL | ng/mL | ng/mL | ng/mL | ng/mL |
| ADAM12 | 197.91 | 89.87 | 40.51 | 0.55 | 57.79 | 16.10 | 70.54 | 70.99 | 180.54 | 8.84 | 47.83 | 22.56 | 23.25 | 416.82 |
| ISM2 | 21.73 | 14.09 | 17.57 | 0.02 | 26.05 | 3.33 | 10.32 | 14.27 | 12.63 | 2.56 | 4.42 | 13.16 | 5.83 | 59.35 |
| PAEP | 260.95 | 214.12 | 152.80 | 91.61 | 464.49 | 358.27 | 141.41 | 161.24 | 95.24 | 243.76 | 304.13 | 22.11 | 334.92 | 351.25 |
| NOTUM | 44.66 | 31.86 | 39.78 | 0.99 | 47.54 | 6.48 | 17.43 | 18.62 | 17.38 | 4.28 | 7.07 | 18.28 | 8.92 | 141.35 |
| CGA | 102.10 | 374.71 | 119.97 | 17.58 | 343.16 | 23.93 | 465.38 | 242.19 | 1482.00 | 66.33 | 190.80 | 447.08 | 68.20 | 390.36 |
| CGB | 156.65 | 453.07 | 150.23 | 23.37 | 447.92 | 40.58 | 654.49 | 434.59 | 2462.06 | 149.58 | 368.55 | 600.87 | 120.42 | 419.18 |
| PAPPA | 180.01 | 32.07 | 97.85 | 0.00 | 93.18 | 14.11 | 15.67 | 16.28 | 24.40 | 2.12 | 10.79 | 12.44 | 16.03 | 309.57 |
| PSG1 | 459.41 | 124.99 | 33.54 | 0.00 | 156.29 | 27.41 | 66.11 | 39.96 | 385.25 | 9.82 | 57.90 | 30.08 | 29.08 | 301.07 |
| PSG2 | 63.19 | 15.51 | 18.79 | 0.08 | 16.82 | 3.68 | 12.75 | 6.70 | 36.70 | 2.31 | 11.96 | 10.53 | 9.38 | 104.98 |
| PSG3 | 9.31 | 24.78 | 10.86 | 3.93 | 22.94 | 2.86 | 19.56 | 15.89 | 15.51 | 13.26 | 9.65 | 17.86 | 10.41 | 33.35 |
| PSG6/9 | 58.85 | 104.01 | 30.57 | 0.00 | 73.70 | 18.23 | 122.59 | 99.55 | 427.33 | 7.82 | 63.03 | 49.08 | 47.68 | 218.12 |
| PSG8/1 | 1529.10 | 400.14 | 152.13 | 0.00 | 596.50 | 99.31 | 271.60 | 139.73 | 1454.40 | 49.48 | 212.33 | 133.94 | 120.87 | 1029.72 |
| PSG9 | 0.04 | 56.04 | 17.15 | 0.07 | 38.80 | 8.17 | 71.24 | 31.79 | 194.83 | 0.10 | 42.87 | 25.33 | 27.43 | 95.34 |
| PSG11 | 48.69 | 19.40 | 3.90 | 0.00 | 26.67 | 1.97 | 25.57 | 8.85 | 68.33 | 8.81 | 11.14 | 2.80 | 9.57 | 101.73 |

| Gene | SAB16 | SAB17 | SAB18 | SAB19 | SAB20 | SAB21 | SAB22 | SAB23 | SAB24 | SAB25 |
|---|---|---|---|---|---|---|---|---|---|---|
| Name | ng/mL | ng/mL | ng/mL | ng/mL | ng/mL | ng/mL | ng/mL | ng/mL | ng/mL | ng/mL |
| ADAM12 | 119.24 | 54.78 | 3.33 | 30.49 | 62.36 | 74.61 | 38.72 | 46.55 | 5.51 | 86.43 |
| ISM2 | 16.51 | 11.66 | 4.22 | 5.17 | 17.55 | 11.55 | 2.42 | 8.25 | 4.71 | 8.35 |
| PAEP | 204.32 | 249.57 | 56.14 | 33.60 | 287.51 | 228.87 | 201.78 | 260.81 | 113.66 | 77.58 |
| NOTUM | 49.09 | 31.69 | 14.57 | 18.19 | 62.99 | 39.26 | 8.29 | 18.54 | 14.64 | 18.10 |
| CGA | 14.00 | 46.55 | 7.86 | 144.91 | 36.07 | 275.04 | 126.38 | 139.37 | 77.59 | 587.82 |
| CGB | 11.91 | 40.59 | 6.79 | 102.95 | 26.33 | 179.75 | 103.78 | 97.65 | 64.40 | 409.85 |
| PAPPA | 271.01 | 93.05 | 39.06 | 51.01 | 151.46 | 52.58 | 28.90 | 55.72 | 1.80 | 97.49 |
| PSG1 | 192.23 | 49.25 | 16.96 | 127.06 | 124.86 | 185.34 | 45.78 | 86.88 | 29.77 | 290.28 |
| PSG2 | 17.69 | 10.95 | 1.96 | 35.23 | 20.81 | 22.75 | 11.19 | 21.86 | 3.44 | 37.35 |
| PSG3 | 9.95 | 20.01 | 0.00 | 9.88 | 16.50 | 21.86 | 15.28 | 28.20 | 6.53 | 15.74 |
| PSG6/9 | 133.14 | 109.69 | 23.96 | 50.82 | 142.27 | 203.95 | 30.37 | 56.25 | 19.02 | 180.61 |
| PSG8/1 | 725.49 | 219.90 | 69.61 | 459.76 | 350.28 | 532.65 | 184.60 | 364.62 | 131.21 | 1035.92 |
| PSG9 | 31.26 | 47.48 | 8.70 | 13.81 | 54.54 | 70.58 | 13.07 | 14.71 | 4.02 | 56.44 |
| PSG11 | 8.88 | 13.73 | 0.44 | 19.09 | 6.31 | 29.06 | 17.52 | 17.56 | 1.26 | 43.46 |

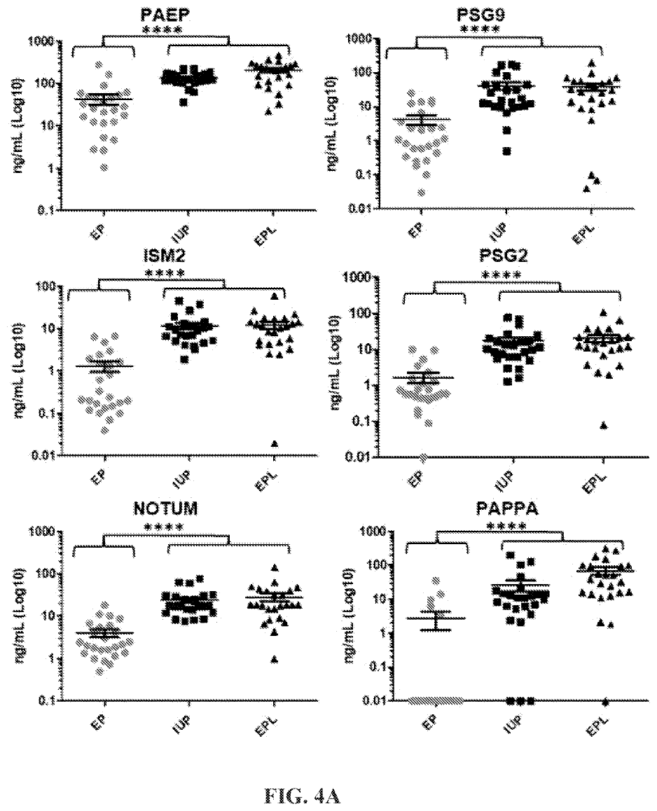

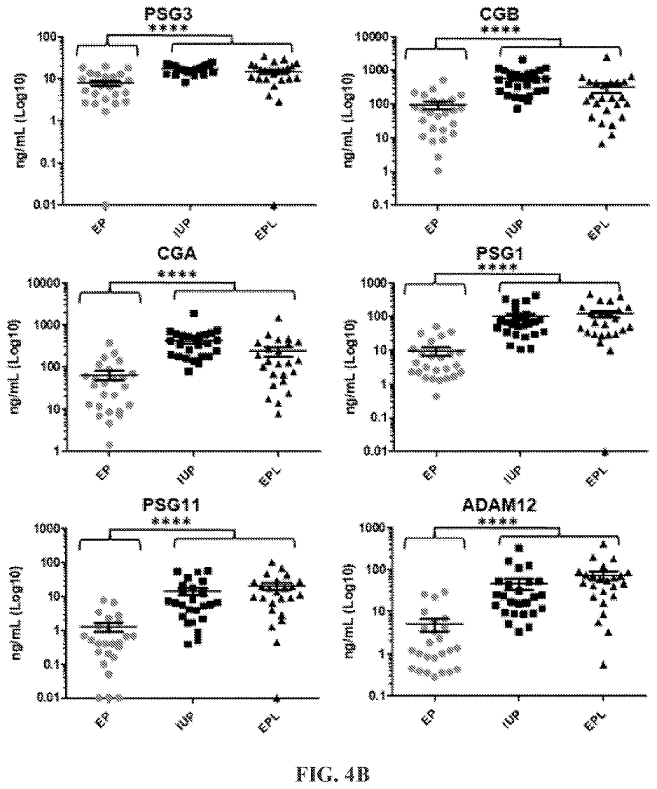

We evaluated 12 unique protein markers (ADAM12, CGA, CGB, ISM2, NOTUM, PAEP, PAPPA, PSG1, PSG2, PSG3, PSG9, and PSG11) and two peptide markers that are shared between two pregnancy specific beta-1-glycoproteins (PSG6/9 and PSG8/1) and found that all markers were significantly different between EP and non-EP patients using Wilcoxon rank sum test (FIG. 4). Likewise, each of the individual biomarkers were excellent at accurately distinguishing EP vs non-EP with AUCs≥0.82. AUCs for all biomarkers were higher in the validation cohort than in the discovery cohort (Table 7).

TABLE 7
Comparisons of AUC between candidate EP vs non-EP biomarkers
identified in the validation cohort (N = 74)

| Gene | | | Std. | 95% confidence | | p- |
|---|---|---|---|---|---|---|
| Name | N | AUC | Error | interval | | Value |
| ISM2 | 74 | 0.941 | 0.029 | 0.8851 | 0.9974 | <0.0001 |
| NOTUM | 74 | 0.939 | 0.029 | 0.8818 | 0.9957 | <0.0001 |
| PSG8/1 | 74 | 0.936 | 0.030 | 0.8776 | 0.9934 | <0.0001 |
| PSG2 | 74 | 0.935 | 0.029 | 0.8773 | 0.9929 | <0.0001 |
| PSG1 | 74 | 0.931 | 0.031 | 0.8703 | 0.991 | <0.0001 |
| ADAM12 | 74 | 0.916 | 0.034 | 0.8491 | 0.9819 | <0.0001 |
| PAEP | 74 | 0.915 | 0.042 | 0.833 | 0.9972 | <0.0001 |
| PSG6/9 | 74 | 0.907 | 0.035 | 0.838 | 0.9758 | <0.0001 |
| PSG11 | 74 | 0.897 | 0.036 | 0.826 | 0.9683 | <0.0001 |
| PAPPA | 74 | 0.894 | 0.040 | 0.8151 | 0.9727 | <0.0001 |
| CGA | 74 | 0.865 | 0.043 | 0.7806 | 0.9484 | <0.0001 |
| PSG9 | 74 | 0.857 | 0.045 | 0.7692 | 0.9443 | <0.0001 |
| PSG3 | 74 | 0.825 | 0.052 | 0.7229 | 0.9269 | <0.0001 |
| CGB | 74 | 0.820 | 0.049 | 0.7232 | 0.916 | <0.0001 |

We further assessed the predictive ability of the 14 markers for EP using univariate logistic regression analysis (Table 8) and multivariable logistic regression with Lasso-selected markers (NOTUM, PAEP, PAPPA, and PSG2) as predictors (Model 1 in Table 1).

TABLE 8
Univariate logistic regression analysis
results for EP vs non-EP (N = 74)

| | | | | | 95% Confidence | |
|---|---|---|---|---|---|---|
| Predictors | Coefficient | SE | z | P > \|z\| | interval | |
| ADAM12 | −0.901 | 0.202 | −4.47 | <0.001 | −1.297 | −0.506 |
| CGA | −0.775 | 0.183 | −4.23 | <0.001 | −1.135 | −0.416 |
| CGB | −0.632 | 0.164 | −3.86 | <0.001 | −0.952 | −0.311 |
| ISM2 | −0.953 | 0.228 | −4.18 | <0.001 | −1.399 | −0.506 |
| NOTUM | −1.567 | 0.360 | −4.35 | <0.001 | −2.273 | −0.861 |
| PAEP | −1.588 | 0.367 | −4.33 | <0.001 | −2.307 | −0.869 |
| PAPPA | −0.362 | 0.070 | −5.16 | <0.001 | −0.499 | −0.224 |
| PSG1 | −0.799 | 0.183 | −4.36 | <0.001 | −1.158 | −0.440 |
| PSG11 | −0.648 | 0.155 | −4.19 | <0.001 | −0.951 | −0.345 |
| PSG2 | −0.991 | 0.216 | −4.58 | <0.001 | −1.415 | −0.567 |
| PSG3 | −0.589 | 0.257 | −2.29 | 0.022 | −1.093 | −0.086 |
| PSG6/9 | −0.707 | 0.174 | −4.05 | <0.001 | −1.048 | −0.365 |
| PSG8/1 | −0.756 | 0.179 | −4.22 | <0.001 | −1.107 | −0.404 |
| PSG9 | −0.473 | 0.115 | −4.11 | <0.001 | −0.699 | −0.248 |

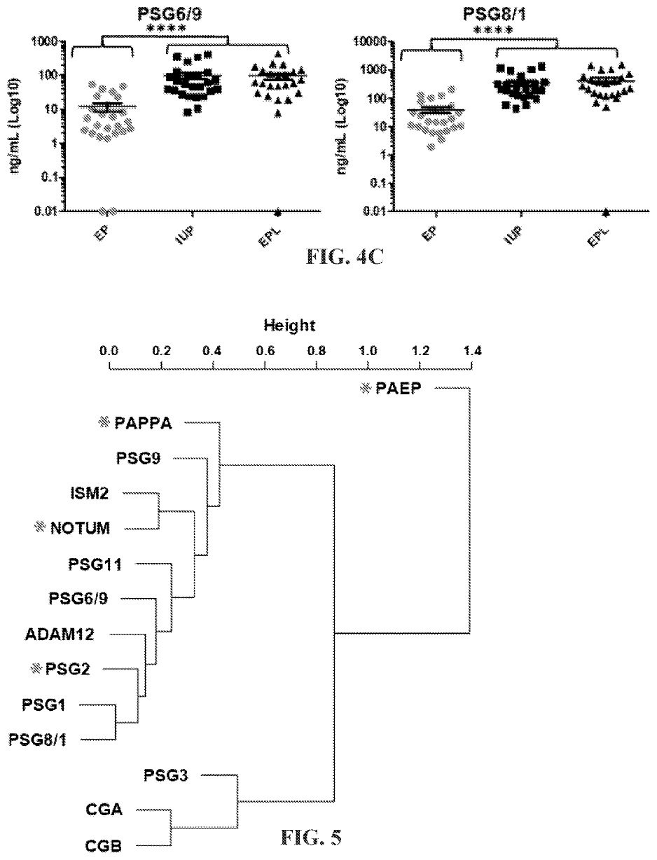

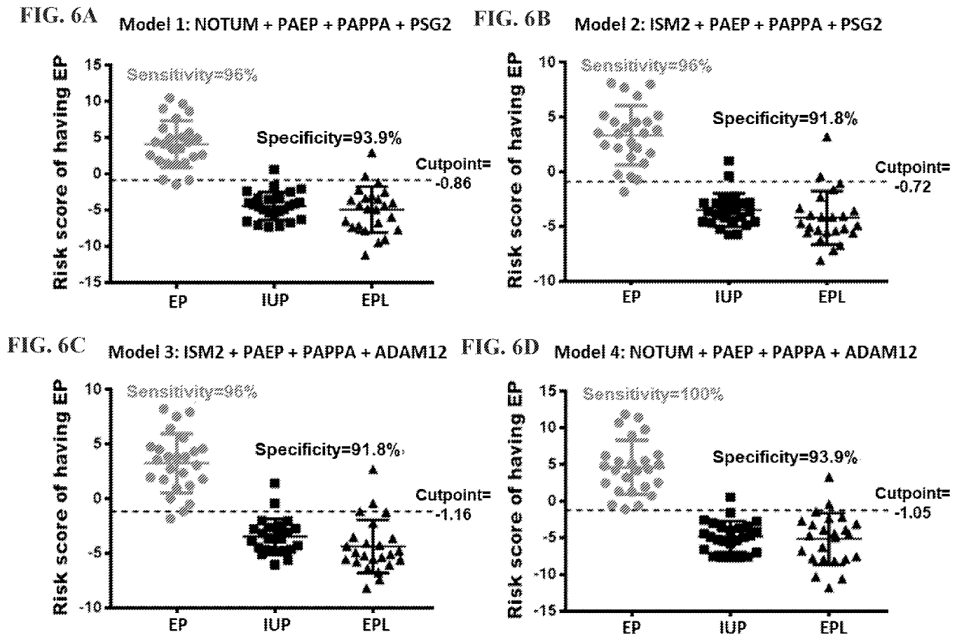

Spearman correlation coefficients indicated that some of the markers correlated with each other (FIG. 5). As expected, due to minimization of multicollinearity, the Lasso-selected protein markers were from different correlation clusters. We subsequently generated additional multivariable logistic regression models in which highly correlated protein markers, specifically NOTUM/ISM2 and PSG2/ADAM12, were exchanged to evaluate the effect on predictive ability for EP (Models 2-4 in Table 1). The multivariable logistic regression models were used to calculate risk scores for EP, and sensitivity and specificity were determined based on optimized cut-points (FIG. 6A-6D). With a cut-point of 0.858, the model using Lasso-selected predictors (Model 1) had a sensitivity of 96% and specificity of 93.9. Model 4, in which PSG2 was exchanged for ADAM12, exhibited the best performance with a sensitivity of 100% and specificity of 93.9% at a risk score cut-point of −1.05. Further, areas under the ROC curves were compared using an algorithm suggested by DeLong, DeLong, and Clarke-Pearson [19] (Table 9).

TABLE 9
Comparisons of AUC between each logistic model with single predictors versus
each multivariable logistic model predicting EP vs non-EP (N = 74)

| | | | | 95% | | Model | Model | Model | Model |
|---|---|---|---|---|---|---|---|---|---|
| | | | | Confidence | | 1: P- | 2: P- | 3: P- | 4: P- |
| Predictors in the model | N | AUC | SE | interval | | value^{a} | value^{a} | value^{a} | value^{a} |
| Model 1: NOTUM + | 74 | 0.986 | 0.010 | 0.967 | 1.000 | reference | N/A | N/A | N/A |
| PAEP + PAPPA + PSG2 | | | | | | | | | |
| Model 2: ISM2 + | 74 | 0.980 | 0.012 | 0.957 | 1.000 | N/A | reference | N/A | N/A |
| PAEP + PAPPA + PSG2 | | | | | | | | | |
| Model 3: ISM2 + | 74 | 0.980 | 0.012 | 0.957 | 1.000 | N/A | N/A | reference | N/A |
| PAEP + PAPPA + ADAM12 | | | | | | | | | |
| Model 4: NOTUM + | 74 | 0.987 | 0.010 | 0.968 | 1.000 | N/A | N/A | N/A | reference |
| PAEP + PAPPA + ADAM12 | | | | | | | | | |
| ISM2 | 74 | 0.941 | 0.029 | 0.885 | 0.997 | 0.051 | 0.071 | 0.078 | 0.046 |
| NOTUM | 74 | 0.939 | 0.029 | 0.882 | 0.996 | 0.049 | 0.073 | 0.078 | 0.044 |
| PSG8/1 | 74 | 0.936 | 0.030 | 0.878 | 0.993 | 0.038 | 0.056 | 0.061 | 0.034 |
| PSG2 | 74 | 0.935 | 0.030 | 0.877 | 0.993 | 0.031 | 0.035 | 0.039 | 0.027 |
| PSG1 | 74 | 0.931 | 0.031 | 0.870 | 0.991 | 0.030 | 0.041 | 0.044 | 0.027 |
| ADAM12 | 74 | 0.916 | 0.034 | 0.849 | 0.982 | 0.018 | 0.022 | 0.023 | 0.017 |
| PAEP | 74 | 0.915 | 0.042 | 0.832 | 0.998 | 0.081 | 0.106 | 0.104 | 0.081 |
| PSG6/9 | 74 | 0.907 | 0.035 | 0.838 | 0.976 | 0.009 | 0.011 | 0.011 | 0.008 |
| PSG11 | 74 | 0.897 | 0.036 | 0.826 | 0.968 | 0.004 | 0.004 | 0.005 | 0.004 |
| PAPPA | 74 | 0.894 | 0.039 | 0.817 | 0.970 | 0.012 | 0.013 | 0.013 | 0.011 |
| CGA | 74 | 0.865 | 0.043 | 0.780 | 0.949 | 0.003 | 0.004 | 0.004 | 0.003 |
| PSG9 | 74 | 0.857 | 0.045 | 0.769 | 0.945 | 0.002 | 0.002 | 0.002 | 0.002 |
| PSG3 | 74 | 0.825 | 0.053 | 0.722 | 0.928 | 0.001 | 0.002 | 0.002 | 0.001 |
| CGB | 74 | 0.820 | 0.049 | 0.723 | 0.916 | 0.000 | 0.001 | 0.001 | 0.000 |

^{a}Single predictor models vs. multivariable model;
N/A: Not Applicable

The AUCs from the multivariable models were all higher than models with single predictors (Table 9). The multivariable models had similar statistical significance by this measure; however, Model 4 again had the best performance with an AUC of 0.987 with 95% CI of 0.968 to 1.0.

### DISCUSSION

In a proteomics discovery of plasma samples from women having IUP, EP, or SAB, we sought to discover biomarkers that could distinguish any of the three pregnancy outcomes from one or more of the other outcomes. We identified 12 promising biomarkers for EP vs non-EP (IUP and EPL) (FIG. 2). For validation in an independent patient cohort using a different MS method, we focused on biomarkers of EP vs non-EP including CGB, CGA, ADAM12, ISM2, NOTUM, PAEP, PAPPA PSG1, PSG2, PSG3, PSG9, PSG11, PSG6/9, and PSG8/1. Most of the markers that we describe, including CGB,CGA, PAPPA, ADAM12, ISM2, NOTUM, and the PSGs are known to be associated with trophoblast function [20-23]. PAEP is secreted from the endometrium and fallopian tube and has an immunomodulatory role in implantation [21]. Hence, it is reasonable that a fetus implanted outside of the uterus would have anomalous levels of these markers compared with normal pregnancy development.

Previous assays identifying EP biomarker were limited by sensitivity because a substantial portion of patients had biomarker levels that fell below the detection limit. Other studies evaluating ADAM12 as a predictor of EP and other adverse pregnancy outcomes have had conflicting results. Yang et al found that ADAM12 maintained low levels in EP and miscarriage compared to normal pregnancies [26], while Horne et al determined that ADAM12 values were increased in EP compared to IUP or miscarriage and, that ADAM12 had limited value as a diagnostic marker for EP when measured in isolation [27]. However, because of the proprietary nature of most commercial antibody assays, the specific domain that was targeted by the antibody and potential cross-reactivity with isoforms and homologs in these studies was unknown.

A critical component of biomarker validation is the accurate, unambiguous quantitation of the target protein isoform while distinguishing homologous family members because an assay that simultaneously measures homologous proteins that do not correlate with the clinical condition will likely reduce accuracy of the diagnosis. This confounding effect is not surprising as related proteins are often associated with distinct structural or functional roles and will not a priori track together with a clinical condition [28, 29]. Assays with poorly defined isoform specificity, have the potential to yield misleading results if multiple related proteins are quantitated together.

The presence of homologous proteins in blood can also complicate protein quantitation using label-free LC-MS/MS analysis because it is not apparent how peptides shared between identified proteins should be distributed between homologs. Specifically, our discovery proteomics analysis of 48 plasma samples identified several candidate biomarkers with ambiguous assignment of shared peptides to highly homologous protein isoforms. This was a particular challenge for the PSG family of proteins. At least nine out of ten possible PSG gene products were detected in plasma, reflecting the abundant nature of these proteins in maternal blood during pregnancy [30, 31]. The extensive sharing of common peptides across isoforms and stochastic detection of many of these peptides limited the accuracy of quantitation of these proteins in the discovery cohort.

To circumvent this complication in the targeted PRM-MS assay, we based protein quantitation on targeted tryptic peptides that were unique to putative diagnostic isoforms plus two shared peptides. Because most single amino acid substitutions can be readily distinguished by MS, targeted MS-based quantitation methods such as multiple reaction monitoring (MRM) and PRM-MS have been used successfully to quantitate specific protein isoforms from cell extracts [32, 33].

For the PSGs, we targeted peptides that were unique to the putative diagnostic isoforms (PSG1, PSG2, PSG3, PSG9, and PSG11), as well as two peptides that were shared between PSG isoforms that were ambiguous from the discovery analysis (PSG1/8 and PSG 6/9). We did not find that these shared peptide markers provided a strong improvement for EP prediction compared to their unique counterparts from PSG1 and PSG9, respectively (Table 7). Therefore, we determined that there was no clear advantage of pursuing these shared markers and de-emphasized them when developing predictive models for EP. Further, it should be noted that, although the individual PSGs are each highly significant candidates, they may be difficult to validate by ELISA assays as part of a clinical biomarker panel unless the assay was rigorously demonstrated to quantify only a single isoform from among the nine isoforms present in patient plasma. In this regard, it should be emphasized that the other four PSG isoforms detected in the discovery study did not correlate with any of the clinical groups and would therefore confound accurate quantitation in assays without sufficient isoform specificity.

It is interesting that all 12 biomarkers from the discovery study validated and had higher AUCs in the larger, independent validation cohort. We primarily attribute this higher performance as biomarkers to the superior quantitative performance of targeted PRM-MS assays compared with LFQ discovery proteomics where the accuracy of protein quantitation is somewhat limited by: 1) variable detection and quantitation of specific peptides across samples, 2) potential inconsistent distribution of shared peptides to different isoforms, and 3) inaccurate distribution of shared peptides to the wrong isoform.

After assessing the predictive ability of the 14 candidate markers both individually and in combinations, we determined that the accuracy of multivariable logistic regression models was higher than those models with single predictors (Table 9). We identified an initial multiple biomarker panel using Lasso feature selection (Model 1: NOTUM, PAEP, PAPPA, PSG2) that has the predictive capacity to identify an EP with high accuracy. We evaluated several alternative panels based on the Lasso-selected model in which highly correlated biomarkers were substituted (i.e., NOTUM with ISM2; PSG2 with ADAM12) and found that they perform similarly to one another (FIG. 5). The biomarker panel with the highest performance was Model 4 (NOTUM, PAEP, PAPPA, ADAM12) with an AUC of 0.987; however, because all four models performed similarly with regard to EP predictive ability, all candidate biomarkers listed in these models as well as other closely correlated biomarkers should be considered in future studies.

The current clinical marker for EP is CGB, and our discovery analysis found that it, along with its fellow chorionic gonadotropin alpha subunit CGA, was among the candidate proteins that distinguished EP from other pregnancy outcomes. However, neither CGB nor CGA were among the features selected by the Lasso analysis and incorporation of CGB, CGA, or the highly correlated marker PSG3 did not significantly improve accuracy of the models. Importantly, we note that all the other tested individual candidate biomarkers and multiprotein panels had higher AUCs than CGB alone (Table 9).

We also explored the minimum number of biomarkers needed for accurate diagnosis of EP by evaluating logistic regression models based on the Lasso-selected model with fewer features.

There is a tradeoff between the performance of a biomarker panel and the feasibility of clinical implementation and further validation studies on multiple patient populations may show that subsets of the four biomarker panels in one or more of the above models may be sufficient for highly accurate diagnosis of EP.

We recognize that MS analysis of proteins is not routinely used in clinical diagnostic laboratories, although its use is becoming more common and it is easier to multiplex compared with ELISA. Our use of a multiplexed, targeted PRM-MS assay was primarily to unambiguously quantify related isoforms for robust biomarker validation.

This study used discovery proteomics to identify 12 plasma protein biomarkers that distinguish EP from either IUP or EPL, and all 12 proteins were validated in a larger, independent patient cohort using a more quantitatively accurate, multiplexed PRM-MS assay. Highly accurate diagnosis of EP could be achieved using a four-protein biomarker panel consisting of NOTUM, PAEP, PAPPA, and ADAM12 with an algorithm combining these biomarkers to calculate an EP risk score having an AUC of 0.987. In addition, several other biomarkers closely correlate with three of these biomarkers and other models that substitute closely clustered biomarkers perform similarly.

In addition to the four-protein models presented here, several other substitutions are feasible. As different combinations of biomarkers may perform somewhat differently in larger follow-up validation studies, these results suggest that further validation studies should focus on nine proteins (PAEP, PAPPA, PSG9, ISM2, NOTUM, PSG11, ADAM12, PSG2, and PSG1) using assays that accurately quantitate specific isoforms in the presence of highly homologous isoforms.

### REFERENCES

1. Seeber B E, Barnhart K T. Suspected ectopic pregnancy. Obstet Gynecol. 2006; 107(2 Pt 1):399-413.
2. Senapati S, Sammel M D, Butts S F, Takacs P, Chung K, Barnhart K T. Predicting first trimester pregnancy outcome: derivation of a multiple marker test. Fertil Steril. 2016; 106(7):1725-32 e3.
3. Ammon Avalos L, Galindo C, Li D K. A systematic review to calculate background miscarriage rates using life table analysis. Birth Defects Res A Clin Mol Teratol. 2012; 94(6):417-23.
4. Jurkovic D, Overton C, Bender-Atik R. Diagnosis and management of first trimester miscarriage. BMJ. 2013; 346:f3676.
5. Whittaker P G, Schreiber C A, Sammel M D. Gestational hormone trajectories and early pregnancy failure: a reassessment. Reprod Biol Endocrinol. 2018; 16(1):95.
6. American College of O, Gynecologists. ACOG Practice Bulletin No. 94: Medical management of ectopic pregnancy. Obstet Gynecol. 2008; 111(6):1479-85.
7. Wang H, Tang H Y, Tan G C, Speicher D W. Data analysis strategy for maximizing high-confidence protein identifications in complex proteomes such as human tumor secretomes and human serum. J Proteome Res. 2011; 10(11):4993-5005.
8. Gerszten R E, Asnani A, Carr S A. Status and prospects for discovery and verification of new biomarkers of cardiovascular disease by proteomics. Circ Res. 2011; 109(4):463-74.
9. Gerszten R E, Carr S A, Sabatine M. Integration of proteomic-based tools for improved biomarkers of myocardial injury. Clin Chem. 2010; 56(2):194-201.
10. Beer L A, Ky B, Barnhart K T, Speicher D W. In-Depth, Reproducible Analysis of Human Plasma Using IgY 14 and SuperMix Immunodepletion. Methods Mol Biol. 2017; 1619:81-101.
11. Goldman A R, Beer L A, Tang H Y, Hembach P, Zayas-Bazan D, Speicher D W. Proteome Analysis Using Gel-LC-MS/MS. Curr Protoc Protein Sci. 2019:e93.
12. Schnatbaum K, Zerweck J, Nehmer J, Wenschuh H, Schutkowski M, Reimer U. SpikeTides™—proteotypic peptides for large-scale MS-based proteomics. Nature Methods. 2011; 8.
13. Cox J, Mann M. MaxQuant enables high peptide identification rates, individualized p.p.b.-range mass accuracies and proteome-wide protein quantification. Nat Biotechnol. 2008; 26(12):1367-72.
14. Geiger T, Wehner A, Schaab C, Cox J, Mann M. Comparative proteomic analysis of eleven common cell lines reveals ubiquitous but varying expression of most proteins. Mol Cell Proteomics. 2012; 11(3):M111 014050.
15. Beer L A, Liu P, Ky B, Barnhart K T, Speicher D W. Efficient Quantitative Comparisons of Plasma Proteomes Using Label-Free Analysis with MaxQuant. Methods Mol Biol. 2017; 1619:339-52.
16. Tyanova S, Temu T, Sinitcyn P, Carlson A, Hein M Y, Geiger T, et al. The Perseus computational platform for comprehensive analysis of (prote) omics data. Nat Methods. 2016; 13(9):731-40.
17. Pino L K, Searle B C, Bollinger J G, Nunn B, MacLean B, MacCoss M J. The Skyline ecosystem: Informatics for quantitative mass spectrometry proteomics. Mass Spectrom Rev. 2020; 39(3):229-44
18. Tusher V G, Tibshirani R, Chu G. Significance analysis of microarrays applied to the ionizing radiation response. Proc Natl Acad Sci USA. 2001; 98(9):5116-21.
19. DeLong E R, DeLong D M, Clarke-Pearson D L. Comparing the areas under two or more correlated receiver operating characteristic curves: a nonparametric approach. Biometrics. 1988; 44(3):837-45.
20. Jeyarajah M J, Jaju Bhattad G, Kelly R D, Baines K J, Jaremek A, Yang F P, et al. The multifaceted role of GCM1 during trophoblast differentiation in the human placenta. Proc Natl Acad Sci USA. 2022; 119(49):e2203071119.
21. Soundravally R, Pooja D. Biomarkers of Ectopic Pregnancy-Present and Future. 2015.
22. Martinez C, Gonzalez-Ramirez J, Marin M E, Martinez-Coronilla G, Meza-Reyna V I, Mora R, et al. Isthmin 2 is decreased in preeclampsia and highly expressed in choriocarcinoma. Heliyon. 2020; 6(10):e05096.
23. Marchand M, Horcajadas J A, Esteban F J, McElroy S L, Fisher S J, Giudice L C. Transcriptomic signature of trophoblast differentiation in a human embryonic stem cell model. Biol Reprod. 2011; 84(6):1258-71.
24. Beer L A, Tang H Y, Sriswasdi S, Barnhart K T, Speicher D W. Systematic discovery of ectopic pregnancy serum biomarkers using 3-D protein profiling coupled with label-free quantitation. J Proteome Res. 2011; 10(3):1126-38.
25. Rausch M E, Beer L, Sammel M D, Takacs P, Chung K, Shaunik A, et al. A disintegrin and metalloprotease protein-12 as a novel marker for the diagnosis of ectopic pregnancy. Fertil Steril. 2011; 95(4):1373-8.
26. Yang J, Wu J, Guo F, Wang D, Chen K, Li J, et al. Maternal serum disintegrin and metalloprotease protein-12 in early pregnancy as a potential marker of adverse pregnancy outcomes. PLoS One. 2014; 9(5):e97284.
27. Horne A W, Brown J K, Tong S, Kaitu'u-Lino T. Evaluation of ADAM-12 as a diagnostic biomarker of ectopic pregnancy in women with a pregnancy of unknown location. PLoS One. 2012; 7(8):e41442.
28. Garcia-Blanco M A, Baraniak A P, Lasda E L. Alternative splicing in disease and therapy. Nat Biotechnol. 2004; 22(5):535-46.
29. Kriventseva E V, Koch I, Apweiler R, Vingron M, Bork P, Gelfand M S, et al. Increase of functional diversity by alternative splicing. Trends Genet. 2003; 19(3):124-8.
30. Moore T, Dveksler G S. Pregnancy-specific glycoproteins: complex gene families regulating maternal-fetal interactions. Int J Dev Biol. 2014; 58(2-4):273-80.
31. Moore T, Williams J M, Becerra-Rodriguez M A, Dunne M, Kammerer R, Dveksler G. Pregnancy-specific glycoproteins: evolution, expression, functions and disease associations. Reproduction. 2022; 163(2):R11-R23.
32. Wang Q, Chaerkady R, Wu J, Hwang H J, Papadopoulos N, Kopelovich L, et al. Mutant proteins as cancer-specific biomarkers. Proc Natl Acad Sci USA. 2011; 108(6):2444-9.
33. Zulak K G, Lippert D N, Kuzyk M A, Domanski D, Chou T, Borchers C H, et al. Targeted proteomics using selected reaction monitoring reveals the induction of specific terpene synthases in a multi-level study of methyl jasmonate-treated Norway spruce (Picea abies). Plant J. 2009; 60(6):1015-30.
34. Tang H Y, Beer L A, Tanyi J L, Zhang R, Liu Q, Speicher D W. Protein isoform-specific validation defines multiple chloride intracellular channel and tropomyosin isoforms as serological biomarkers of ovarian cancer. J Proteomics. 2013; 89:165-78.
35. Mueller M D, Raio L, Spoerri S, Ghezzi F, Dreher E, Bersinger N A. Novel placental and nonplacental serum markers in ectopic versus normal intrauterine pregnancy. Fertil Steril. 2004; 81(4):1106-11.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A diagnostic reagent or kit for use in diagnosing an ectopic pregnancy in a mammalian subject comprising:
(a) a ligand that that binds to a peptide or protein selected from the group consisting of:
i. disintegrin and metalloproteinase domain-containing protein 12 (ADAM12),
ii. Glycoprotein hormones alpha chain (CGA),
iii. Choriogonadotropin subunit beta (CGB),
iv. Isthmin-2 (ISM2),
v. protein notum homolog (NOTUM),
vi. Glycodelin (PAEP),
vii Pappalysin-1 (PAPPA0,
viii. pregnancy specific beta-1 glycoprotein, isoform 1 (PSG1),
ix. pregnancy specific beta-1 glycoprotein, isoform 2 (PSG2),
x. pregnancy specific beta-1 glycoprotein, isoform 3 (PSG3),
xi. pregnancy specific beta-1 glycoprotein, isoform 9 (PSG9),
xii. pregnancy specific beta-1 glycoprotein, isoform 11 (PSG11),
xiii. pregnancy specific beta-1 glycoprotein, isoforms 6 and 9 (PSG6/9), and
xiv. pregnancy specific beta-1 glycoprotein, isoforms 8 and 1 (PSG8/1), or
(b) a combination of ligand (a), wherein each ligand binds to a different peptide or protein (i) through (xiv);
wherein at least one of the ligands is associated with a detectable label or with a substrate.

2. The reagent or kit of claim 1, wherein the kit consists of (v), (vi), (vii) and (ix).

3. The reagent or kit of claim 1, wherein the kit consists of (iv), (vi), (vii) and (ix).

4. The reagent or kit of claim 1, wherein the kit consists of (iv), (vi), (vii) and (i).

5. The reagent or kit of claim 1, wherein the kit consists of (v), (vi), (vii) and (i).

6. The reagent or kit according to claim 1, which comprises a substrate upon which said ligand is immobilized.

7. The reagent or kit according to claim 1, wherein said ligands or said expression products are proteins or peptides.

8. The reagent or kit according to claim 8, wherein said ligand is an antibody or fragment thereof.

9. The reagent or kit according to claim 1, wherein the ligand is associated with a detectable label.

10. The reagent or kit according to claim 1, comprising a microarray, a microfluidics card, a chip or a chamber.

11. A method for diagnosing an ectopic pregnancy in a female mammalian subject comprising:
(a) measuring in a biological fluid sample of the subject the expression level of a protein or peptide fragment thereof selected from the group consisting of:
i. disintegrin and metalloproteinase domain-containing protein 12 (ADAM12),
ii. Glycoprotein hormones alpha chain (CGA),
iii. Choriogonadotropin subunit beta (CGB),
iv. Isthmin-2 (ISM2),
v. protein notum homolog (NOTUM),
vi. Glycodelin (PAEP),
vii Pappalysin-1 (PAPPA0,
viii. pregnancy specific beta-1 glycoprotein, isoform 1 (PSG1),
ix. pregnancy specific beta-1 glycoprotein, isoform 2 (PSG2),
x. pregnancy specific beta-1 glycoprotein, isoform 3 (PSG3),
xi. pregnancy specific beta-1 glycoprotein, isoform 9 (PSG9),
xii. pregnancy specific beta-1 glycoprotein, isoform 11 (PSG11),
xiii. pregnancy specific beta-1 glycoprotein, isoforms 6 and 9 (PSG6/9), and
xiv. pregnancy specific beta-1 glycoprotein, isoforms 8 and 1 (PSG8/1),
xv. a combination of any of (i) through (xiv) and
(b) comparing said subject's expression level of the selected protein or peptide fragment with the level of the same protein or peptide in the biological fluid of a reference or control female mammalian subject having a normal intrauterine pregnancy (IUP),
wherein changes in expression of the subject's selected protein or peptide fragment from those of the reference or control correlates with a diagnosis of ectopic pregnancy.

12. The method according to any one of claim 11, wherein said change in expression level of each said selected protein or peptide fragment comprises an upregulation in comparison to said reference or control or a downregulation in comparison to said reference or control.

13. The method of claim 11, wherein the proteins consist of (v), (vi), (vii) and (ix).

14. The method of claim 11, wherein the proteins consist of (iv), (vi), (vii) and (ix).

15. The method of claim 11, wherein the proteins consist of (iv), (vi), (vii) and (i).

16. The method of claim 11, wherein the proteins consist of (v), (vi), (vii) and (i).


SEQUENCE LISTING (36 sequences)

SEQ ID NO: 1 - protein, 9 aa, Homo sapiens
NHPEVLNIR

SEQ ID NO: 2 - protein, 13 aa, Homo sapiens
VNSAGDPYGNCGK

SEQ ID NO: 3 - protein, 13 aa, Homo sapiens
FGFGGSTDSGPIR

SEQ ID NO: 4 - protein, 8 aa, Homo sapiens
TPEVTPLR

SEQ ID NO: 5 - protein, 14 aa, Homo sapiens
TRPCGYGCTATETR

SEQ ID NO: 6 - protein, 9 aa, Homo sapiens
LDIYQPTAR

SEQ ID NO: 7 - protein, 10 aa, Homo sapiens
FDTTPWILCK

SEQ ID NO: 8 - protein, 19 aa, Homo sapiens
ACTDNPLEEEYLAQLQEAK

SEQ ID NO: 9 - protein, 20 aa, Homo sapiens
VHITSLLPTPEDNLEIVLHR

SEQ ID NO: 10 - protein, 9 aa, Homo sapiens
HLWYLLDLK

SEQ ID NO: 11 - protein, 17 aa, Homo sapiens
VLVEDDEIMQGFIRSUM

SEQ ID NO: 12 - protein, 9 aa, Homo sapiens
LGYPAIQVR

SEQ ID NO: 13 - protein, 18 aa, Homo sapiens
DVPASFAPACLSHEIIIR

SEQ ID NO: 14 - protein, 9 aa, Homo sapiens
SHWTDVQVK

SEQ ID NO: 15 - protein, 7 aa, Homo sapiens
AYPTPLR

SEQ ID NO: 16 - protein, 11 aa, Homo sapiens
VTVMGGFKSUM

SEQ ID NO: 17 - protein, 17 aa, Homo sapiens
VLQGVLPALPQVVCNYR

SEQ ID NO: 18 - protein, 10 aa, Homo sapiens
DHPLTCDDPR

SEQ ID NO: 19 - protein, 11 aa, Homo sapiens
SPAVITGLYDK

SEQ ID NO: 20 - protein, 9 aa, Homo sapiens
AYLDVNELK

SEQ ID NO: 21 - protein, 7 aa, Homo sapiens
FQLPGQK

SEQ ID NO: 22 - protein, 9 aa, Homo sapiens
IHPSYTNYR

SEQ ID NO: 23 - protein, 16 aa, Homo sapiens
FQQSGQNLFIPQITTK

SEQ ID NO: 24 - protein, 15 aa, Homo sapiens
NSATGEESSTSLTVK

SEQ ID NO: 25 - protein, 10 aa, Homo sapiens
IGLLPLLNPT

SEQ ID NO: 26 - protein, 12 aa, Homo sapiens
EDAGSYTLHIVK

SEQ ID NO: 27 - protein, 16 aa, Homo sapiens
VSAPSGTGHLPGLNPL

SEQ ID NO: 28 - protein, 9 aa, Homo sapiens
TLYLFGVTK

SEQ ID NO: 29 - protein, 9 aa, Homo sapiens
TLFLLGVTK

SEQ ID NO: 30 - protein, 14 aa, Homo sapiens
LPIPYITINNLNPR

SEQ ID NO: 31 - protein, 16 aa, Homo sapiens
SNPVILNVLYGPDLPR

SEQ ID NO: 32 - protein, 8 aa, Homo sapiens
LFIPQITR

SEQ ID NO: 33 - protein, 14 aa, Homo sapiens
VSGPCHGDLTESQS

SEQ ID NO: 34 - protein, 18 aa, Homo sapiens
YTAGPYECEIWNSGSASR

SEQ ID NO: 35 - protein, 9 aa, Homo sapiens
LFIPQITPK

SEQ ID NO: 36 - protein, 16 aa, Homo sapiens
VIAPPGLGTFAFNNPT